SEQUENCE LISTING (36 sequences)

SEQ ID NO: 1 - DNA, 1242 bp, Mus musculus
atgactccctccatctcatggggtctactgcttctggcaggcctgtgttgcctggtccccagctttctggctgaggatgttcaggagacagacacctcccagaaggatcagtccccagcctcccatgagatcgctacaaacctgggagactttgcaatcagcctataccgggagctggtccatcagtccaacacttccaacatcttcttctccccagtgagcattgccacagcctttgctatgctctccctagggagcaagggtgacactcacacgcagatcctagagggcctgcagttcaacctcacacaaacatcggaggctgacatccacaagtccttccaacacctcctccaaaccctcaacagaccagacagtgagctgcagttgagcacaggcaatggcctctttgtcaacaatgacctgaagctggtggagaagtttctggaagaggccaagaaccattatcaggcagaagtcttctctgtcaactttgcagagtcagaggaggccaagaaagtgattaatgattttgtggagaagggaacccaaggaaagatagttgaggcagtgaaagaactggaccaagacacagttttcgccctgggcaattacattctttttaaaggcaaatggaagaagccattcgatcctgagaacactgaagaagctgagttccacgtggacaagtccaccacggtgaaggtgcccatgatgaccctctcgggcatgcttgatgtgcaccattgcagcacactctccagctgggtgctgctgatggattacgcgggcaacgccagtgctgtcttcctcctgcccgaagatgggaagatgcagcatctggagcaaactctcaacaaggagctcatctctaagatcctgctaaacaggcgcagaaggttagtccagatccatatccccagactgtccatctctggagaatataacttgaagacactcatgagtccactgggcatcacccggatcttcaacaatggggctgacctctccggaatcacagaggagaatgctcccctgaagctcagcaaggctgtgcataaggctgtgctgaccatcgatgagacaggaacagaagctgcagcagctacagtctttgaagccgttcctatgtctatgccccctatcctgcgcttcgaccaccctttcctttttataatatttgaagaacacactcagagccccatctttgtgggaaaagtggtagatcccacacataaatga

SEQ ID NO: 2 - DNA, 297 bp, Mus musculus
atgcctacagagactgagagatgcattgagtccctgattgctgttttccaaaagtacagcgggaaggatggaaacaacactcaactctccaaaactgaattcctttccttcatgaacacagagctggctgccttcacaaagaaccagaaggatcctggtgtccttgaccgcatgatgaagaagctggacctcaactgtgacgggcagctagatttccaagagtttctcaacctcattggtggcttagctatagcgtgccatgattctttcatccaaacttcccagaagcgaatctaa

SEQ ID NO: 3 - DNA, 20 bp, Mus musculus
ttgaagaagccattcgatcc

SEQ ID NO: 4 - DNA, 20 bp, Mus musculus
tgaaaaggaaagggtggtcg

SEQ ID NO: 5 - DNA, 20 bp, Mus musculus
atgcctacagagactgagag

SEQ ID NO: 6 - DNA, 22 bp, Mus musculus
gattcgcttctgggaagtttgg

SEQ ID NO: 7 - DNA, 1378 bp, Mesocricetus auratus
atcagctctgggacaggcaagctaaaaatgaagccctccatctcatgggggatcctgctgctggcaggcctgtgctgcctggtccccagcttcctggctgaggatgcccaggagacagatgcctccaagcaggatcaggagcaccaagcctgctgtaagatcgctccaaatttggcagacttttccttcaacctataccgggagctggtccatcagtccaatacgaccaacatcttcttctctcctgtgagcattgccacagcctttgctatgctctctctgggcaccaagggtgtcactcacacccagattctagagggcctggggttcaacctcacagaaatagccgaggctgaggtccacaaaggcttccataacctcctccagaccttcaacaggccagacaatgagcttcagctgaccacaggcaatggcctgttcatccacaacaatctaaagctggtggataagttcctggaagaggtcaagaacgattaccactcggaagccttctctgtcaacttcacagactcagaagaggccaagaaagtgatcaacggttttgtggagaagggaacccaaggaaagatagttgatttagtgaaggaccttgacaaagacacagttcttgccctggtgaattacattttctttaaaggcaagtggaagaagcccttcgatgcagacaacactgaggaagctgacttccacgtggacaagaccaccacggtgaaggtgcccatgatgagccgcctgggcatgtttgacgtgcactatgttagcactctgtccagctgggtgctgctgatggattacctgggcaacgccactgccatcttcatcctacctgatgatggcaagatgcagcatctggagcaaactctcaacaaggaaatcattggcaagttcctgaaggacagacacacaaggtcagccaatgtacacttccccaaactgtccatctctggaacctataacttgaagacagccctggatccgctgggcatcacccaggtcttcagcaatggggccgacctttctgggatcacagaggatgttcccctgaagcttggcaaggctgtgcataaggctgtgctgaccatcgatgagagagggacggaagctgcaggggccacatttatggaaatcatccccatgtctgtgccccctgaggtgaactttaacagccctttcattgccataatatatgatagacagacagcaaagagccccctctttgtgggaaaagtggtggatcccacacgttaatcacaattctcagtcagatgtcatcttttctggattgggtcccctccccagtgacattaaacacaggctgtcctggcccacccatgcctgagtgcttctgcaaatgctc

SEQ ID NO: 8 - DNA, 1345 bp, Homo sapiens
acatgtaatcgacaatgccgtcttctgtctcgtggggcatcctcctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctaaaccagccagacagccagctccagctgaccaccggcaatggcctgttcctcagcgagggcctgaagctagtggataagtttttggaggatgttaaaaagttgtaccactcagaagccttcactgtcaacttcggggatcacgaagaggccaagaaacagatcaacgattacgtggagaagggtactcaagggaaaattgtggatttggtcaaggagcttgacagagacacagtttttgctctggtgaattacatcttctttaaaggcaaatgggagagaccttttgaagtcaaggacaccgaggacgaggacttccacgtggaccaggtgaccaccgtgaaggtccctatgatgaagcgtttaggcatgtttaacatccagcactgtaagaagctgtccagctgggtactgctaatgaaatacctgggcaatgccaccgccatcttcttcctacctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaggggactgaagctgctggggccatgtttttagaggccataccaatgtctatccccccagaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctgga

SEQ ID NO: 9 - DNA, 1353 bp, Oryctolagus cuniculus
atatcatctccccatctttgttcctgccaccagccctgggcactgagtcctggacaatgccaccctctgtctctcgggcgctcctcctgctggccggcctgggctgcctgctgcccggcttcctggccgacgaggcccaggagacagccgtttccagccatgagcaggaccgcccagcctgccacaggatcgccccgagcctggttgagttcgccctcagcctgtaccgggaggtggcccgcgagtccaacaccaccaatatcttcttctccccggtgagcatcgccctggcctttgccatgctctccctgggggccaagggggacacccacacccaggtcctggagggcctgaagttcaacctcacggagacggccgaggcccagatccacgacggcttccggcacctcctgcacaccgtcaacaggcccgacagcgagctgcagctggccgccggcaacgccctggtcgtcagcgagaacctgaagctgcagcacaagtttctagaagacgccaagaacctgtaccagtccgaagccttcctcgtcgacttcagggaccccgagcaggccaagaccaagatcaacagccacgtggagaaggggacccgagggaagatcgtggacttggtgcaagagctggacgcccgcacactgcttgccctggtgaactacgttttcttcaaagggaagtgggagaagcccttcgagcccgagaacaccaaggaagaggacttccacgtggacgccacgaccacggtgcgggtgcccatgatgtcgcgcctgggcatgtatgtgatgttccactgtagcacgctggccagcacggtcgtgctgatggactacaagggcaacgccacggccctcttcctcctgcccgacgaggggaagctgcagcacctggagcacacgctcaccacggagctcatcgccaagttcctggcaaaaagcagcttcaggtctgtcacggtccgttttcccaaactctccatttctggaacctacgacctgaaacccctcctgggcaaactgggcatcacccaggtcttcagcgacaacgcggacctctcggggatcacggagcaggaagctctgaaggtgtcccaggccctgcacaaggtggtgctgaccatcgacgagagagggaccgaagctgccggggccacatttgtggaatacgtactctattctatgccccaaagggtcacctttgacaggcccttcctctttgtcatctacagtcatgaggtcaagagtcccctcttcgtggggaaagtggtggatcccacccaacactaagaccccaccgcagcacattaaagctctgagctgccctcccagggggcagcccctc

SEQ ID NO: 10 - DNA, 1306 bp, Rattus norvegicus
gctccatctcacgggggctcctgcttctggcagccctgtgttgcctggcccccagcttcctggctgaggatgcccaggaaaccgatacctcccagcaggaccagagtccaacctaccgtaagatttcttcaaacctggcagactttgccttcagcctataccgggagctggtccatcaatccaatacatccaacatcttcttctcccctatgagcatcaccacagccttcgccatgctctccctggggagcaagggtgacactcgcaaacagattctagagggcctggagttcaacctcacacagatacctgaggctgacatccacaaggccttccatcacctcctccaaactctcaacaggccagacagtgagctgcagctgaacacaggcaatggcctctttgtcaacaagaatctgaagctggtggagaagtttctggaagaggtcaagaacaattaccactcagaagccttctctgtcaactttgccgactcagaagaggctaagaaagtaattaatgattatgtagagaagggaacccaaggaaagatagttgatttgatgaaacagctggacgaagacacggtttttgccctggtgaattacattttctttaaaggcaagtggaagaggccattcaatcctgagcacactagggatgctgactttcacgtagacaagtccaccacagtgaaggtgcccatgatgaaccgcctgggcatgtttgacatgcactattgcagcacactgtccagctgggtgctgatgatggattacctgggcaacgccactgccatcttcctcctgcccgatgatggcaagatgcagcatctggagcaaactctcaccaaggatctcatttcccggttcctgctaaacaggcaaacaaggtcagccattctctacttccccaaactgtccatctctggaacctataacttgaagacactcctgagctcactgggcatcacccgggtcttcaacaatgatgctgatctctctggaatcacagaggatgcccccctgaagcttagccaggctgtgcataaggctgtgctgaccttagatgagaggggaacagaggctgcaggagccactgtggtggaggccgtccccatgtctctgccccctcaagtgaagttcgaccaccctttcattttcatgatagttgaatcagaaactcagagccccctctttgtgggaaaagtgatagatcccacacgttaatcactgtcctcagaagtcacatcccttctggatcgggtccccttcctaataatattaaactcaggctggcctggcct

SEQ ID NO: 11 - DNA, 1334 bp, Ovis aries
cgataatggcactctccatcacacggggccttctgctgctggcagccctgtgctgcctggcccccacctccctggctggggttctccaaggacacgctgtccaagagacagatgatacagcccaccaggaagcagcctgccacaagattgcccccaacctggccaactttgccttcagcatataccacaagttggcccatcagtccaataccagcaacatcttcttctccccagtgagcatcgcttcagcctttgcgatgctttccctgggagccaagggcaacactcacactgagatcctggagggcctgggtttcaacctcactgagctagcagaggctgagatccacaaaggctttcagcatcttctccacaccctcaaccagccaaaccaccagctgcaactgaccaccggcaatggtctgttcatcaatgagagtgcaaagctagttgatacgtttttggaggatgtcaagaatctgcatcactccaaagccttctccatcaacttcagggatgctgaggaggccaagaagaagatcaatgattatgtagagaagggaagccatggaaaaattgtggatttggtaaaggatcttgaccaagacacagtttttgctctggtcaattacatatcctttaaaggaaaatgggagaagcccttcgaggtcgagcacaccacggagagggacttccacgtgaatgagcaaaccaccgtgaaggtgcccatgatgaaccgcctgggcatgtttgacctccactactgtgacaagctcgccagctgggtgctgctgctggactacgtgggcaacgtcaccgcctgcttcatcctgcccgacctcgggaaactgcagcagctggaagacaagctcaacaacgaactcctcgccaagttcctggaaaagaaatatgcaagttctgccaatttacatttgcccaaactgtccatttctgaaacgtacgatctgaaaactgtcctgggtgaactgggcatcaacagggtcttcagcaacggggctgacctctcagggatcaccgaggaacagcctctgatggtgtccaaggcgctccacaaggctgcgctgaccattgatgagaaagggacagaagctgctggggccacgtttctggaagctatccccatgtcccttcccccagacgtcgagttcaacagacccttcctctgcatcctctacgacagaaacaccaagtctcccctcttcgtgggaaaggtggtgaatcccacccaagcctaagtgcctctcggggttcagctttcccctcccaggccaggtccccttcttccctccatggcattaaaggataactgacct

SEQ ID NO: 12 - DNA, 183 bp, Oryctolagus cuniculus
ttcgccgtgttccagaagtacgctggaaaggatgggcacagcgtcaccctctccaagaccgagttcctgtcctttatgaacacagagctggctgccttcacaaagaaccagaaggaccccggcgtcctcgaccgcatgatgaagaaattggacctcaacagtgacgggcagctggatttccaa

SEQ ID NO: 13 - DNA, 428 bp, Canis lupus familiaris
gcacgaggtctctgattgctgttttccagaagtttgctggaaaggagggtaacaactgcacactctccaagacagagttcctaaccttcatgaatacagaactggctgccttcacaaagaaccagaaggaccctggtgtccttgaccgcatgatgaagaaactggacctcaactctgatgggcagctggatttccaagaatttcttaatcttattggtggcatggccatagcttgccatgactcctttacaaggtctccccatttccggaagtaaatcggaggggttcctgggcctggcctccagaccacctctttccttcaaaacagcttcccaatcatcacatccttctcacatcctacacagacctgagcccacagtgtccaccaccctgtgcaggccagtcctgctggtagtgaataaagcaat

SEQ ID NO: 14 - DNA, 282 bp, Homo sapiens
atgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtag

SEQ ID NO: 15 - DNA, 270 bp, Rattus norvegicus
atggcaactgaactggagaaggccttgagcaacgtcattgaagtctaccacaattattctggtataaaagggaatcaccatgccctctacagggatgacttcaggaaaatggtcactactgagtgccctcagtttgtgcagaataaaaataccgaaagcttgttcaaagaattggacgtcaatagtgacaacgcaattaacttcgaagagttccttgcgttggtgataagggtgggcgtggcagctcataaagacagccacaaggagtaa

SEQ ID NO: 16 - DNA, 300 bp, Sus scrofa
atggcaaaaagacccacagagactgagcgttgcattgaatctctgattgctattttccaaaagcatgctggaagggacggtaacaacacgaaaatctccaagaccgagttcctaattttcatgaatacagagctggctgccttcacacagaaccagaaagaccctggtgtccttgaccgcatgatgaagaaattggacctcgactctgatgggcagctagatttccaagaatttcttaatcttattggcggcctggccatagcttgccatgactcctttattaagtctacccagaagtaa

SEQ ID NO: 17 - protein, 16 aa, Mus musculus
CQSPIFVGKVVDPTHK

SEQ ID NO: 18 - protein, 16 aa, Mus musculus
CIACHDSFIQTSQKRI

SEQ ID NO: 19 - protein, 413 aa, Mus musculus
MTPSISWGLLLLAGLCCLVPSFLAEDVQETDTSQKDQSPASHEIATNLGDFAISLYRELVHQSNTSNIFFSPVSIATAFAMLSLGSKGDTHTQILEGLQFNLTQTSEADIHKSFQHLLQTLNRPDSELQLSTGNGLFVNNDLKLVEKFLEEAKNHYQAEVFSVNFAESEEAKKVINDFVEKGTQGKIVEAVKELDQDTVFALGNYILFKGKWKKPFDPENTEEAEFHVDKSTTVKVPMMTLSGMLDVHHCSTLSSWVLLMDYAGNASAVFLLPEDGKMQHLEQTLNKELISKILLNRRRRLVQIHIPRLSISGEYNLKTLMSPLGITRIFNNGADLSGITEENAPLKLSKAVHKAVLTIDETGTEAAAATVFEAVPMSMPPILRFDHPFLFIIFEEHTQSPIFVGKVVDPTHK

SEQ ID NO: 20 - protein, 98 aa, Mus musculus
MPTETERCIESLIAVFQKYSGKDGNNTQLSKTEFLSFMNTELAAFTKNQKDPGVLDRMMKKLDLNCDGQLDFQEFLNLIGGLAIACHDSFIQTSQKRI

SEQ ID NO: 21 - DNA, 1434 bp, Artificial Sequence
  Synthetic construct
nnnnnnnnnnnnnnnnnnnnnnnnnnnnnnnnnnnnnnnnnnnnnnnnnnnnganaatgcnccctccatctcatgggggctcctgctgctggcaggcctgtgctgcctggtccccagcttcctggctgaggatnnnnnnnnnnnnnnngcccaggagacagatnnnacctcccagcaggatcaggancncccagcctgccataagatcgctccaaacctggcagactttgccttcagcctataccgggagctggtccatcagtccaataccaccaacatcttcttctccccagtgagcatcgccacagcctttgcnatgctctccctggggaccaagggtgacactcacacncagatcctggagggcctggagttcaacctcacagagatagcngaggctgagatccacaaaggcttccagcacctcctccanaccctcaacaggccagacagtgagctgcagctgaccaccggcaatggcctgttcgtcaacgagaatctgaagctggtggataagtttctggaagaggtcaagaacctttaccactcagaagccttctctgtcaacttcggggactcagaggaggccaagaaagtgatcaatgattatgtggagaagggaacccaaggaaagatagttgatttggtgaaggagcttgacnaagacacagtttttgccctggtgaattacattttctttaaaggcaagtgggagaagcccttcgatgccgagaacactgaggaagctgacttccacgtggacaagnccaccacggtgaaggtgcccatgatgaaccgcctgggcatgtttgacatgcactattgtagcacgctgtccagctgggtgctgctgatggattacctgggcaacgccactgccatcttcctcctgcccgatgatgggaagctgcagcatctggagcaaactctcaccaagganctcatcgccaagttcctggaaaacagacacacaaggtctgccaatntccatttccccaaactgtccatttctggaacctatgacttgaagacagtcctgggtccactgggcatcacccgggtcttcagcaatggggctgacctctcngggatcacagaggannntgcncccctgaagctntgcaaggctgtgcataaggctgtgctgaccatcgatgagagagggacagaagctgcaggggccacatttntggaagccgtccccatgtctatgccccctgaggtgaagttcgacagccctttccttttcataatatttgaanaacagannnccaagagtcccctctttgtgggaaaagtggtggatcccacccatnaataactgcctctcggnnnacatcncatcccttcnngnccnggtcccctnnnnnccccnatgacattaaannnnggctgncctggnnnnnnnnnnnnnnnnnnnnnnnnnnnnnnnnn

SEQ ID NO: 22 - DNA, 460 bp, Artificial Sequence
  Synthetic construct
nnnnnnnnnatgncnacngagnnngagannngcatgaantctctgattgctgttttccanaagtatgctggaaaggangggaacaacnntaccctctccaagactgagttcctgaccttcatgaatacagagctggctgccttcacaaagaaccagaaggaccctggtgtccttgaccgcatgatgaagaaattggacctcaactgtgatggngcagctagatttccaagagtttcttaatctnattggnggcntggccatagcntgccatgantcnttnannnannctacccanaannngaagtaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 23 - protein, 382 aa, Rattus norvegicus
APSHGGSCFWQPCVAWPPASWLRMPRKPIPPSRTRVQPTVRFLQTWQTLPSAYTGSWSINPIHPTSSSPLASPQPSPCSPWGARVTLANRFRAWSSTSHRYLRLTSTRPSITSSKLSTGQTVSCSTQAMASLSTRISWWRSFWKRSRTITTQKPSLSTLPTQKRLRKLMIMRREPKERLINSWTKTRFLPWITFSLKASGRGHSILSTLGMLTFTTSPPQRCPTAWACLTCTIAAHCPAGCWITWATPLPSSSCPMMARCSIWSKLSPRISFPGSCTGKQGQPFSTSPNCPSLEPITRHSAHWASPGSSTMMLISLESQRMPPSLARLCIRLCPMRGEQRLQEPLWWRPSPCLCPLKSSTTLSFSLNQKLRAPSLWEKIPHV

SEQ ID NO: 24 - protein, 417 aa, Homo sapiens
MPSSVSWGILLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDHEEAKKQINDYVEKGTQGKIVDLVKELDRDTVFALVNYIFFKGKWERPFEVKDTEDEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 25 - protein, 416 aa, Ovis aries
MALSITRGLLLLAALCCLAPTSLAGVLQGHAVQETDDTAHQEAACHKIAPNLANFAFSIYHKLAHQSNTSNIFFSPVSIASAFAMLSLGAKGNTHTEILEGLGFNLTELAEAEIHKGFQHLLHTLNQPNHQLQLTTGNGLFINESAKLVDTFLEDVKNLHHSKAFSINFRDAEEAKKKINDYVEKGSHGKIVDLVKDLDQDTVFALVNYISFKGKWEKPFEVEHTTERDFHVNEQTTVKVPMMNRLGMFDLHYCDKLASWVLLLDYVGNVTACFILPDLGKLQQLEDKLNNELLAKFLEKKYASSANLHLPKLSISETYDLKTVLGELGINRVFSNGADLSGITEEQPLMVSKALHKAALTIDEKGTEAAGATFLEAIPMSLPPDVEFNRPFLCILYDRNTKSPLFVGKVVNPTQA

SEQ ID NO: 26 - protein, 413 aa, Mesocricetus auratus
MKPSISWGILLLAGLCCLVPSFLAEDAQETDASKQDQEHQACCKIAPNLADFSFNLYRELVHQSNTTNIFFSPVSIATAFAMLSLGTKGVTHTQILEGLGFNLTEIAEAEVHKGFHNLLQTFNRPDNELQLTTGNGLFIHNNLKLVDKFLEEVKNDYHSEAFSVNFTDSEEAKKVINGFVEKGTQGKIVDLVKDLDKDTVLALVNYIFFKGKWKKPFDADNTEEADFHVDKTTTVKVPMMSRLGMFDVHYVSTLSSWVLLMDYLGNATAIFILPDDGKMQHLEQTLNKEIIGKFLKDRHTRSANVHFPKLSISGTYNLKTALDPLGITQVFSNGADLSGITEDVPLKLGKAVHKAVLTIDERGTEAAGATFMEIIPMSVPPEVNFNSPFIAIIYDRQTAKSPLFVGKVVDPTR

SEQ ID NO: 27 - protein, 413 aa, Oryctolagus cuniculus
MPPSVSRALLLLAGLGCLLPGFLADEAQETAVSSHEQDRPACHRIAPSLVEFALSLYREVARESNTTNIFFSPVSIALAFAMLSLGAKGDTHTQVLEGLKFNLTETAEAQIHDGFHNLLQTFNRPDNELQLTTGNGLFIHNNLKLVDKFLEEVKNDYHSEAFSVNFTDSEEAKKVINSHVEKGTRGKIVDLVQELDARTLLALVNYVFFKGKWEKPFEPENTKEEDFHVDATTTVRVPMMSRLGMYVMFHCSTLASTVVLMDYKGNATALFLLPDEGKLQHLEHTLTTELIAKFLAKSSFRSVTVRFPKLSISGTYDLKPLLGKLGITQVFSDNADLSGITEQEALKVSQALHKVVLTIDERGTEAAGATFVEYVLYSMPQRVTFDRPFLFVIYSHEVKSPLFVGKVVDPTQH

SEQ ID NO: 28 - protein, 424 aa, Artificial Sequence
  Synthetic construct
MXPSISXGLLLLAGLCCLVPSFLAEDXQXXXXXETDXSXHDQDXPACHKIAPNLADFAFSLYRELAHQSNTTNIFFSPVSIATAFAMLSLGTKGDTHTQILEGLXFNLTETAEAEIHKGFQHLLXTLNRPDSELQLTTGNGLFISEXLKLVDKFLEDVKNLYHSEAFSVNFXDSEEAKKINDFVEKGTQGKIVDLVKELDKDTVLALVNYIFFKGKWEKPFEVENTEEXDFHVDXTTTVKVPXXXXMMSRLGMFDVHHCSTLSSWVLLMDYLGNATAIFILPDDGKLQHLEQTLNXELIAKFLXNRXXRSASLHLPKLSISGTYDLKTLLGXLGITRVFSNGADLSGITEEXXPLKLSKAVHKAVLTIDEKGTEAAGATFLEAIPMSMPPEVXFNRPFLFIIYDXNTKSXPLFVGKVVDPTQXX

SEQ ID NO: 29 - protein, 90 aa, Canis lupus familiaris
TRSLIAVFQKFAGKEGNNCTLSKTEFLTFMNTELAAFTKNQKDPGVLDRMMKKLDLNSDGQLDFQEFLNLIGGMAIACHDSFTRSPHFRK

SEQ ID NO: 30 - protein, 61 aa, Oryctolagus cuniculus
FAVFQKYAGKDGHSVTLSKTEFLSFMNTELAAFTKNQKDPGVLDRMMKKLDLNSDGQLDFQ

SEQ ID NO: 31 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 32 - protein, 89 aa, Rattus norvegicus
MATELEKALSNVIEVYHNYSGIKGNHHALYRDDFRKMVTTECPQFVQNKNTESLFKELDVNSDNAINFEEFLALVIRVGVAAHKDSHKE

SEQ ID NO: 33 - protein, 99 aa, Sus scrofa
MAKRPTETERCIESLIAIFQKHAGRDGNNTKISKTEFLIFMNTELAAFTQNQKDPGVLDRMMKKLDLDSDGQLDFQEFLNLIGGLAIACHDSFIKSTQK

SEQ ID NO: 34 - protein, 101 aa, Artificial Sequence
  Synthetic construct
XXXMXTEXEKXIXSLIAVFQKYAGKDGNNXXLSKTEFLSFMNTELAAFTKNQKDPGVLDRMMKKLDLNSDGQLDFQEFLNLIGGLAIACHDSFXKSSXKXX

SEQ ID NO: 35 - DNA, 1242 bp, Mus musculus
tcatttatgtgtgggatctaccacttttcccacaaagatggggctctgagtgtgttcttcaaatattataaaaaggaaagggtggtcgaagcgcaggatagggggcatagacataggaacggcttcaaagactgtagctgctgcagcttctgttcctgtctcatcgatggtcagcacagccttatgcacagccttgctgagcttcaggggagcattctcctctgtgattccggagaggtcagccccattgttgaagatccgggtgatgcccagtggactcatgagtgtcttcaagttatattctccagagatggacagtctggggatatggatctggactaaccttctgcgcctgtttagcaggatcttagagatgagctccttgttgagagtttgctccagatgctgcatcttcccatcttcgggcaggaggaagacagcactggcgttgcccgcgtaatccatcagcagcacccagctggagagtgtgctgcaatggtgcacatcaagcatgcccgagagggtcatcatgggcaccttcaccgtggtggacttgtccacgtggaactcagcttcttcagtgttctcaggatcgaatggcttcttccatttgcctttaaaaagaatgtaattgcccagggcgaaaactgtgtcttggtccagttctttcactgcctcaactatctttccttgggttcccttctccacaaaatcattaatcactttcttggcctcctctgactctgcaaagttgacagagaagacttctgcctgataatggttcttggcctcttccagaaacttctccaccagcttcaggtcattgttgacaaagaggccattgcctgtgctcaactgcagctcactgtctggtctgttgagggtttggaggaggtgttggaaggacttgtggatgtcagcctccgatgtttgtgtgaggttgaactgcaggccctctaggatctgcgtgtgagtgtcacccttgctccctagggagagcatagcaaaggctgtggcaatgctcactggggagaagaagatgttggaagtgttggactgatggaccagctcccggtataggctgattgcaaagtctcccaggtttgtagcgatctcatgggaggctggggactgatccttctgggaggtgtctgtctcctgaacatcctcagccagaaagctggggaccaggcaacacaggcctgccagaagcagtagaccccatgagatggagggagtcat

SEQ ID NO: 36 - DNA, 297 bp, Mus musculus
ttagattcgcttctgggaagtttggatgaaagaatcatggcacgctatagctaagccaccaatgaggttgagaaactcttggaaatctagctgcccgtcacagttgaggtccagcttcttcatcatgcggtcaaggacaccaggatccttctggttctttgtgaaggcagccagctctgtgttcatgaaggaaaggaattcagttttggagagttgagtgttgtttccatccttcccgctgtacttttggaaaacagcaatcagggactcaatgcatctctcagtctctgtaggcat

(19) United States
(12) Patent Application Publication
(10) Pub. No.: US 2009/0155797 A1
(43) Pub. Date: Jun. 18, 2009

(54) Methods For Generating Enhanced Antibody-Producing Cell Lines With Improved Growth Characteristics

(76) Inventors: Luigi Grasso, Bala Cynwyd, PA (US); J. Bradford Kline, Norristown, PA (US); Nicholas C. Nicolaides, Garnett Valley, PA (US); Philip M. Sass, Audubon, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/277,743

(22) Filed: Nov. 25, 2008

Related U.S. Application Data
(63) Continuation of application No. 10/624,631, filed on Jul. 21, 2003, now abandoned.
(60) Provisional application No. 60/397,027, filed on Jul. 19, 2002.

(51) Int. Cl. C12Q 1/68; C12N 5/10; C12N 5/00

(52) U.S. Cl. 435/6; 435/325; 435/252.3; 435/419; 435/354; 435/353; 435/363; 435/375

(57) ABSTRACT

The use of mismatch repair (MMR) defective antibody producer cells offers a method to generate subclone variants with elevated protein production such as antibodies. Using MMR defective cells and animals, new cell lines and animal varieties with novel and useful properties such as enhanced protein production can be generated more efficiently than by relying on the natural rate of mutation. These methods are useful for generating genetic diversity within host cells to alter endogenous genes that can yield increased titer levels of protein production. By employing this method, two genes were discovered whose suppressed expression is associated with enhanced antibody production. Suppressed expression of these genes by a variety of methods leads to increased antibody production for manufacturing as well as strategies for modulating antibody production in immunological disorders. Moreover, the suppression of these two genes in host cells can be useful for generating universal high titer protein production lines.

### CROSS-REFERENCE TO RELATED APPLICATIONS

This application is a continuation of U.S. application Ser. No. 10/624,631, filed Jul. 21, 2003, which claims the benefit of U.S. Provisional Application No. 60/397,027, filed Jul. 19, 2002, each of which is hereby incorporated by reference in its entirety.

### TECHNICAL FIELD OF THE INVENTION

The invention is related to the area of antibody and recombinant protein production. In particular, it is related to the field of mutagenesis, gene discovery and recombinant gene expression.

### BACKGROUND OF THE INVENTION

The use of antibodies to block the activity of foreign and/or endogenous polypeptides provides an effective and selective strategy for treating the underlying cause of disease. In particular is the use of monoclonal antibodies (MAb) as effective therapeutics such as the FDA approved ReoPro (Glaser, V. (1996) “Can ReoPro repolish tarnished monoclonal therapeutics?” Nat. Biotechnol. 14:1216-1217), an anti-platelet MAb from Centocor; Herceptin (Weiner, L. M. (1999) “Monoclonal antibody therapy of cancer” Semin. Oncol. 26:43-51), an anti-Her2/neu MAb from Genentech; and Synagis (Saez-Llorens, X. E., et al. (1998) “Safety and pharmacokinetics of an intramuscular humanized monoclonal antibody to respiratory syncytial virus in premature infants and infants with bronchopulmonary dysplasia” Pediat. Infect. Dis. J. 17:787-791), an anti-respiratory syncytial virus MAb produced by Medimmune.

Standard methods for generating MAbs against candidate protein targets are known by those skilled in the art. Briefly, primates as well as rodents, such as mice or rats, are injected with a purified antigen in the presence of adjuvant to generate an immune response (Shield, C. F., et al. (1996) “A cost-effective analysis of OKT3 induction therapy in cadaveric kidney transplantation” Am. J. Kidney Dis. 27:855-864). Animals with positive immune sera are sacrificed and splenocytes are isolated. Isolated splenocytes are fused to myelomas to produce immortalized cell lines that are then screened for antibody production. Positive lines are isolated and characterized for antibody production. The direct use of rodent-derived MAbs as human therapeutic agents were confounded by the fact that human anti-rodent antibody (HARA) responses occurred in a significant number of patients treated with the rodent-derived antibody (Khazaeli, M. B., et al., (1994) “Human immune response to monoclonal antibodies” J. Immunother. 15:42-52). In order to circumvent the problem of HARA, the grafting of the complementarity determining regions (CDRs), which are the critical motifs found within the heavy and light chain variable regions of the immunoglobulin (Ig) subunits making up the antigen binding domain, onto a human antibody backbone found these chimeric molecules to retain their binding activity to antigen while lacking the HARA response (Emery, S. C., and Harris, W. J. “Strategies for humanizing antibodies” In: ANTIBODY ENGINEERING C. A. K. Borrebaeck (Ed.) Oxford University Press, N.Y. 1995. pp. 159-183. A common problem that exists during the “humanization” of rodent-derived MAbs (referred to hereon as HAb) is the loss of binding affinity due to conformational changes in the three-dimensional structure of the CDR domain upon grafting onto the human Ig backbone (U.S. Pat. No. 5,530,101 to Queen et al.). To overcome this problem, additional HAb vectors are usually needed to be engineered whereby inserting or deleting additional amino acid residues within the framework region and/or within the CDR coding region itself in order to recreate high affinity HAbs (U.S. Pat. No. 5,530,101 to Queen et al.). This process is a very time consuming procedure that involves the use of expensive computer modeling programs to predict changes that may lead to a high affinity HAb. In some instances the affinity of the HAb is never restored to that of the MAb, rendering them of little therapeutic use.

A problem that exists in antibody engineering is the generation of stable high yielding producer cell lines that is required for manufacturing of the molecule for clinical materials. Several strategies have been adopted in standard practice by those skilled in the art to circumvent this problem. One method is the use of Chinese Hamster Ovary (CHO) cells transfected with exogenous Ig fusion genes containing the grafted human light and heavy chains to produce whole antibodies or single chain antibodies, which are a chimeric molecule containing both light and heavy chains that form an antigen-binding polypeptide (Reff, M. E. (1993) “High-level production of recombinant immunoglobulins in mammalian cells” Curr. Opin. Biotechnol. 4:573-576).

Another method employs the use of human lymphocytes derived from transgenic mice containing a human grafted immune system or transgenic mice containing a human Ig gene repertoire. Yet another method employs the use of monkeys to produce primate MAbs, which have been reported to lack a human anti-monkey response (Neuberger, M., and Gruggermann, M. (1997) “Monoclonal antibodies: Mice perform a human repertoire” Nature 386:25-26). In all cases, the generation of a cell line that is capable of generating sufficient amounts of high affinity antibody poses a major limitation for producing sufficient materials for clinical studies. Because of these limitations, the utility of other recombinant systems such as plants are currently being explored as systems that will lead to the stable, high-level production of humanized antibodies (Fiedler, U., and Conrad, U. (1995) “High-level production and long-term storage of engineered antibodies in transgenic tobacco seeds” Bio/Technology 13:1090-1093).

A method for generating genetically altered host cells either surrogate mammalian cells such as but not limited to SP20, NS0, CHO, etc. that are capable of secreting increased amounts of antibody will provide a valuable method for creating cell hosts for product development as well as allow for the generation of reagents useful for the discovery of downstream genes whose altered structure or expression levels when altered result in enhanced MAb production. The invention described herein is directed to the creation of genetically altered cell hosts with increased antibody production via the blockade of MMR that can in turn be used to screen and identify altered gene loci for directed alteration and generation of high titer production strains.

The invention facilitates the generation of high titer production of cell lines with elevated levels of antibody production for manufacturing as well as use for target discovery of genes involved in over-production of antibodies either a the gene expression level, processing level or secretion level. Other advantages of the present invention are described in the examples and figures described herein.

### SUMMARY OF THE INVENTION

The invention provides methods for generating genetically altered antibody producing cell hosts in vitro and in vivo, whereby the cell exhibits enhanced production, processing and/or extracellular secretion of a given antibody molecule, immunoglobulin (Ig) chain or a polypeptide containing regions homologous to an Ig domain(s). The invention also provides methods of employing such high titer antibody producer cells for gene discovery to identify genes involved in regulating enhanced immunoglobulin expression, stability, processing and/or secretion. One method for identifying cells with increased antibody production is through the screening of mismatch repair (MMR) defective cells producing antibody, Ig light and/or heavy chains or polypeptides with Ig domains.

The antibody producing cells suitable for use in the invention include, but are not limited to rodent, primate, human hybridomas or lymphoblastoids; mammalian cells transfected and expressing exogenous Ig light and/or heavy chains or chimeric single chain molecules; plant cells, yeast or bacteria transfected and expressing exogenous Ig light or heavy chains, or chimeric single chain molecules.

Thus, the invention provides methods for making a hypermutable antibody producing cells by inhibiting mismatch repair in cells that are capable of producing antibodies. The cells that are capable of producing antibodies include cells that naturally produce antibodies, and cells that are engineered to produce antibodies through the introduction of immunoglobulin heavy and/or light chain encoding sequences.

The invention also provides methods of making hypermutable antibody producing cells by introducing a dominant negative mismatch repair (MMR) gene such as PMS2 (preferably human PMS2), MLH1, PMS1, MSH2, or MSH2 into cells that are capable of producing antibodies as described in U.S. Pat. No. 6,146,894 to Nicolaides et al. The dominant negative allele of a mismatch repair gene may be a truncation mutation of a mismatch repair gene (preferably a truncation mutation at codon 134, or a thymidine at nucleotide 424 of wild-type PMS2). The invention also provides methods in which mismatch repair gene activity is suppressed. This may be accomplished, for example, using antisense molecules directed against the mismatch repair gene or transcripts; RNA interference, polypeptide inhibitors such as catalytic antibodies, or through the use of chemical inhibitors such as those described in PCT publication No. WO 02/054856.

The invention also provides methods for making a hypermutable antibody producing cells by introducing a nucleotide (e.g., antisense or targeting knock-out vector) or genes encoding for polypeptides (e.g., dominant negative MMR gene allele or catalytic antibodies) into fertilized eggs of animals. These methods may also include subsequently implanting the eggs into pseudo-pregnant females whereby the fertilized eggs develop into a mature transgenic animal as described in U.S. Pat. No. 6,146,894 to Nicolaides et al. These nucleotide or polypeptide inhibitors may be directed to any of the genes involved in mismatch repair such as, for example, PMS2, MLH1, MLH3, PMS1, MSH2, MSH3, or MSH6.

The invention also provides homogeneous compositions of cultured, hypermutable, mammalian cells that are capable of producing antibodies and contain a defective mismatch repair process, wherein the cells contain a mutation in at least one gene responsible for higher production of antibodies in the cells. The defects in MMR may be due to any defect within the mismatch repair genes that may include, for example, PMS2, MLH1, MLH3, PMS1, MSH2, MSH3, MSH4 or MSH6. The cells of the culture may contain dominant negative MMR gene alleles such as PMS2 or MLH3 (Nicolaides, N. C. et al (1998) A Naturally Occurring hPMS2 Mutation Can Confer a Dominant Negative Mutator Phenotype. Mol. Cell. Biol. 18:1635-1641. 1997; U.S. Pat. No. 6,146,894; Lipkin S M, Wang V, Jacoby R, Banerjee-Basu S, Baxevanis A D, Lynch H T, Elliott R M, Collins F S. (2000) MLH3: a DNA mismatch repair gene associated with mammalian microsatellite instability. Nat. Genet. 24:27-35).

The invention also provides methods of introducing immunogloblin genes into mismatch repair defective cells and screening for subclones that yield higher titer antibody or Ig polypeptides than observed in the pool or as compared to mismatch proficient cells.

The invention also provides methods for generating a mutation(s) in a gene(s) affecting antibody production in an antibody-producing cell by culturing the mismatch repair defective cell and testing the cell to determine whether the cell harbors mutations within the gene of interest, such that a new biochemical feature (e.g., over-expression, intracellular stability, processing and/or secretion of antibody or immunoglobulin gene products) is generated. The testing may include analysis of the steady state RNA or protein levels of the immunoglobulin gene of interest, and/or analysis of the amount of secreted protein encoded by the immunoglobulin gene of interest. The invention also embraces mismatch repair defective immunoglobulin producing prokaryotic and eukaryotic transgenic cells made by this process, including cells from rodents, non-human primates and humans.

The invention also provides methods of reversibly altering the hypermutability of an antibody producing cell. In the case that MMR deficiency is due to the use of a dominant negative MMR gene allele, whereby the gene is in an inducible vector containing a dominant negative allele of a mismatch repair gene operably linked to an inducible promoter, the cell is treated with an inducing agent to express the dominant negative mismatch repair gene (such as but not limited to PMS2 (preferably human PMS2), MLH1, MLH3 or PMS1). Alternatively, the cell may be MMR defective due to inactivation of an endogenous MMR gene such as but not limited to PMS1, PMS2, MLH1, MLH3, MSH2, MSH3, MSH4, MSH6. In this instance, expression vectors capable of complementing one of the defective MMR gene subunits is introduced and stably expressed in the cell thereby restoring the MMR defective phenotype using methods as previously described in the literature (Koi M, Umar A, Chauhan D P, Cheman S P, Carethers J M, Kunkel T A, Boland C R. (1994) “Human chromosome 3 corrects mismatch repair deficiency and microsatellite instability and reduces N-methyl-N′-nitro-N-nitrosoguanidine tolerance in colon tumor cells with homozygous hMLH1 mutation” Cancer Res. 15:4308-12).

In another embodiment, the cells may be rendered capable of producing antibodies by co-transfecting a preselected immunoglobulin light and/or heavy chain gene or cDNA of interest. The immunoglobulin genes of the hypermutable cells, or the proteins produced by these methods may be analyzed for desired properties, and genetic hypermutability induction may be stopped such that the genetic stability of the host cell is restored using methods described above.

The invention also provides methods for employing a mismatch repair defective cell line whereby the line is transfected with an immunoglobulin full length or partial light, heavy chain genes either individually or in combination.

The invention also provides methods for generating genetically altered cell lines that express enhanced amounts of an antigen binding polypeptide. These antigen-binding polypeptides may be, for example, Fab domains of antibodies. The methods of the invention also include methods for generating genetically altered cell lines that secrete enhanced amounts of an antigen binding polypeptide. The cell lines are rendered hypermutable by inhibition of mismatch repair that provide an enhanced rate of genetic hypermutation in a cell producing antigen-binding polypeptides such as antibodies. Such cells include, but are not limited to surrogate cell lines such as baby hamster kidney (BHK), Chinese hamster ovary (CHO), NSO, SPO/2, as well as rodent and human derived hybridomas. Expression of enhanced amounts of antigen binding polypeptides may be through enhanced transcription or translation of the polynucleotides encoding the antigen binding polypeptides, through enhanced intracellular stability or through the enhanced secretion of the antigen binding polypeptides.

The invention also provides a composition of matter and method of use of two genes discovered by the above methods whose expression when suppressed in antibody producer cells results in enhanced antibody production. Using comparative gene expression analysis between parental and hypermutable MAb over-producer cell lines, two genes (SEQ ID NO:1 and SEQ ID NO:2) were identified in an over-producer subclone to have significantly lower expression than the parental precursor line. Antisense expression constructs were prepared and antisense vectors were introduced into parental and assayed for enhanced MAb production. Blockade of expression of both genes resulted in significantly higher MAb production.

The invention also provides methods for inhibiting the expression and/or function of said genes by methods used by those skilled in the art such as but not limited to antisense technology incorporating RNA, DNA and/or modified versions thereof (e.g., thioated, etc.); RNA interference; DNA knockout methods of somatic cells or pluripotent cells; ribozymes; intracellular and/or extracellular antibodies; dominant negative protein inhibitors that effect expression and/or function; pharmacologic saturation of substrates or ligands that may bind the gene products; molecules of biological or chemical basis that can effect the gene expression profiles of said genes.

The invention also provides methods for screening for molecules that can affect the biological effect(s) of the genes by employing biological or chemical molecules that can regulate the gene's pathway to regulate immunoglobulin production. These can be through the use of introducing pharmacological amounts of natural or synthetic substrates, or molecules that can deregulate the biological production and/or activity of the genes.

The invention also provides methods for screening for natural subclone variants that may lack expression of said genes by analyzing subclones of pools of cells producing antibody or Ig heavy and/or light chain genes. Screening methods can be carried out by monitoring for protein production in growth medium of cell clones, intracellular protein or message steady state levels or by screening genomic structure of the gene's locus.

The invention also provides methods for screening for inhibitors of expression and/or biological function of said genes to suppress immunoglobulin production in immunological disease states whereby suppressed expression of various immunoglobulin subtypes can relieve, suppress or cure such pathological disease states.

These and other aspects of the invention are provided by one or more of the embodiments described below.

One embodiment of the invention is a method for using mismatch repair defective cells to identify genes involved in enhanced antibody expression, stability, or secretion. MMR activity of a cell is suppressed gene and the cell becomes hypermutable as a result of defective MMR. The cell is grown. The cell is tested for the expression of new phenotypes where the phenotype is enhanced expression, processing and/or secretion of an antibody or Ig heavy and/or light chain polypeptide or derivative thereof.

In another embodiment of the invention, a mismatch repair defective cell overproducing antibody, immunoglobulins, or derivatives thereof is genetically analyzed in comparison to parental cell line to identify altered genes involved in enhanced antibody or immunoglobulin expression, stability, processing, and/or secretion. Altered genetic loci or loci with altered expression are then validated by introducing altered genes or altering gene expression in parental line to confirm role in enhanced immunoglobulin and/or MAb production.

Yet another embodiment of the invention is the discovery and composition of matter of two genes (SEQ ID NO:1 and SEQ ID NO:2) whose suppressed expression results in enhanced antibody production. Expression analysis of said genes are found to be significantly lower in over-producer sublines as compared to parental lines. Said genes expression are suppressed in parental lines and lines are screened for antibody production. Lines with inhibited expression of genes have enhanced antibody production. Thus, the invention also comprises cell lines for expressing antibody molecules or fragments thereof comprising a defect in at least one of the two genes (alpha-1-antitrypsin (SEQ ID NO:1) and monocyte-activating polypeptide I (SEQ ID NO:2)) such that expression of the gene is suppressed or inhibited. The cell lines may be bacterial, yeast, plant or mammalian cells including, but not limited to rabbit cells, rodent cells (e.g., mouse, rat, hamster), and primate cells (including human cells).

Yet another embodiment of the invention is the use of biological or chemical inhibitors of said gene products or natural ligands/substrates of said gene products to regulate the production of antibody, immunoglobulin or derivatives thereof for use in manufacturing.

Yet another embodiment of the invention is a method for screening the expression of said genes (SEQ ID NO:1 and SEQ ID NO:2) or homologs in subclones of cells from pools of antibody or immunoglobulin light and/or heavy chain producing cells to identify clones with reduced protein expression for development of high-titer production lines.

Yet another embodiment of the invention is the use of biological or chemical inhibitors of said gene products or natural ligands/substrates of said gene products to regulate the production of antibody, immunoglobulin or derivatives thereof for use in regulating immunoglobulin production in disease states such as but not limited to immunological disorders.

These and other embodiments of the invention provide the art with methods that can generate enhanced mutability in prokaryotic and eukaryotic cells and animals as well as providing prokaryotic and eukaryotic cells and animals harboring potentially useful mutations for the large-scale production of antibodies, immunoglobulins and derivatives thereof. Further, the invention provides useful compositions for the production of high titers of antibodies. Finally, the invention provides the art with composition of matter of two genes and there respective homologs, whose regulation can result in the increase of antibody production for use in developing strains for manufacturing as well as devising rational screening methods to identify regulators of the said genes for the treatment of immunological disorders involving hyper or hypo immunoglobulin states.

### BRIEF DESCRIPTION OF THE DRAWINGS

FIG. 1 shows the generation of MMR-defective clones with enhanced steady state antibody levels. An ELISA was carried out measuring antibody yields from 5 day old cultures of 10,000 cells from MMR defective H34 hybridoma clones with enhanced antibody titer yields (>500 ngs/ml) within the conditioned medium as compared to the parental H6 cell line. Lane 1: fibroblast cells (negative control); Lane 2: H6 cell; Lane 3: H34 high titer line.

FIG. 2 shows expression Analysis of Immunoglobulin Enhancer Genes. RT-PCR validating the reduced expression of AAT (panel A) and EMAPI (panel B). RNAs were reverse transcribed from H6 parental and H34 enhanced producer clones and PCR amplified for AAT (panel A), EMAPI (panel B), and dihydrofolate reductase (DHFR) (panel C) which served as control. Samples were amplified for varying cycles to measure steady-state expression. The minus lane was RNA process without reverse transcriptase which served as a negative control.

FIGS. 3A-D shows the structure of immunoglobulin enhancer genes and alignments thereof. Nucleotide and protein sequence of the alpha-1-antitrypsin (mouse nucleotide sequence: SEQ ID NO: 1; reverse complement SEQ ID NO: 35; mouse amino acid sequence: SEQ ID NO: 19; hamster nucleotide sequence: SEQ ID NO: 7; hamster amino acid sequence: SEQ ID NO: 26; human nucleotide sequence: SEQ ID NO: 8; human amino acid sequence: SEQ ID NO: 24; rabbit nucleotide sequence: SEQ ID NO: 9; rabbit amino acid sequence: SEQ ID NO: 27; rat nucleotide sequence: SEQ ID NO: 10; rat amino acid sequence: SEQ ID NO: 23; sheep nucleotide sequence: SEQ ID NO: 11; sheep amino acid sequence: SEQ ID NO: 25; consensus nucleotide sequence: SEQ ID NO: 21; consensus amino acid sequence: SEQ ID NO: 28) and endothelial monocyte-activating polypeptide I (mouse nucleotide sequence: SEQ ID NO: 2; reverse complement SEQ ID NO: 36; mouse amino acid sequence: SEQ ID NO: 20; rabbit nucleotide sequence: SEQ ID NO: 12; rabbit amino acid sequence: SEQ ID NO: 30; dog nucleotide sequence: SEQ ID NO: 13; dog amino acid sequence: SEQ ID NO: 29; human nucleotide sequence: SEQ ID NO: 14; human amino acid sequence: SEQ ID NO: 31; rat nucleotide sequence: SEQ ID NO: 15; rat amino acid sequence: SEQ ID NO: 32; pig nucleotide sequence: SEQ ID NO: 16; pig amino acid sequence: SEQ ID NO: 33; consensus nucleotide sequence: SEQ ID NO: 22; consensus amino acid sequence: SEQ ID NO: 34) gene products.

FIG. 4 shows the use of alpha-1-anti-trypsin antibodies to screen for high-titer antibody producer strains. Supernatant was isolated from H6 parental (lane 1); H34 over-producer strains (lane 2); or H6 high titer producer cells expressing anti-AAT and anti-EMAP and probed for anti-alpha-1-anti-trypsin. As shown by arrow, a robust extracellular production of alpha-1-anti-trypsin is observed in the low antibody producer line while very little is present in supernatants of high producer strains.

### DETAILED DESCRIPTION OF THE INVENTION

The reference works, patents, patent applications, and scientific literature, including accession numbers to GenBank database sequences that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Standard reference works setting forth the general principles of recombinant DNA technology known to those of skill in the art include Ausubel et al. CURRENT PROTOCOLS IN MOLECULAR BIOLOGY, John Wiley & Sons, New York (1998); Sambrook et al MOLECULAR CLONING: A LABORATORY MANUAL, 2D ED., Cold Spring Harbor Laboratory Press, Plainview, N.Y. (1989); Kaufman et al., Eds., HANDBOOK OF MOLECULAR AND CELLULAR METHODS IN BIOLOGY AND MEDICINE, CRC Press, Boca Raton (1995); McPherson, Ed., DIRECTED MUTAGENESIS: A PRACTICAL APPROACH, IRL Press, Oxford (1991).

Methods have been discovered for developing high antibody-producing cells by employing the use of cells or animals with defects in their mismatch repair (MMR) process that in turn results in increased rates of spontaneous mutation by reducing the effectiveness of DNA repair. MMR defective cells or animals are utilized to develop new mutations in a gene of interest. The use of MMR defective cells for production of antibody, immunoglobulin (Ig) gene or derivatives thereof, including cells such as hybridomas; mammalian, plant, yeast or bacterial cells transfected with genes encoding for Ig light and heavy chains or derivatives, can result in subclones that have enhanced production of antibody, immunoglobulin or derivative polypeptides. The process of MMR, also called mismatch proofreading, is carried out by protein complexes in cells ranging from bacteria to mammalian cells (Muller A, Fishel R. (2002) “Mismatch repair and the hereditary non-polyposis colorectal cancer syndrome (HNPCC)” Cancer Invest. 20:102-9). A MMR gene is a gene that encodes for one of the proteins of such a mismatch repair complex. Although not wanting to be bound by any particular theory of mechanism of action, a MMR complex is believed to detect distortions of the DNA helix resulting from non-complementary pairing of nucleotide bases. The non-complementary base on the newer DNA strand is excised, and the excised base is replaced with the appropriate base, which is complementary to the older DNA strand. In this way, cells eliminate many mutations that occur as a result of mistakes in DNA replication.

Dominant negative alleles or inactivation of both alleles by site-specific gene mutation of a given MMR gene can cause a MMR defective phenotype. An example of a dominant negative allele of a MMR gene is the human gene hPMS2-134, which carries a truncating mutation at codon 134. The mutation causes the product of this gene to abnormally terminate at the position of the 134th amino acid, resulting in a shortened polypeptide containing the N-terminal 133 amino acids. Such a mutation causes an increase in the rate of mutations, which accumulate in cells after DNA replication. Expression of a dominant negative allele of a mismatch repair gene results in impairment of mismatch repair activity, even in the presence of the wild-type allele. Any allele which produces such effect can be used in this invention. Dominant negative alleles of a MMR gene can be obtained from the cells of humans, animals, yeast, bacteria, or other organisms. Such alleles can be identified by screening cells for defective MMR activity. Moreover, inactivation of both copies of a given MMR gene can also lead to defective MMR. Cells from animals or humans with cancer can be screened for defective mismatch repair. Cells from colon cancer patients may be particularly useful. Genomic DNA, cDNA, or mRNA from any cell encoding a MMR protein can be analyzed for variations from the wild type sequence. Dominant negative alleles or inactivated alleles of a MMR gene can also be created artificially, for example, by producing variants of the hPMS2-134 allele or other MMR genes. Various techniques of site-directed mutagenesis can be used. The suitability of such alleles, whether natural or artificial, for use in generating hypermutable cells or animals can be evaluated by testing the mismatch repair activity caused by the allele in the presence of one or more wild-type alleles, to determine if it is a dominant negative allele or inactivated allele.

Methods used by those skilled in the art can also be employed to suppress the endogenous activity of a MMR gene resulting in enhanced DNA hypermutability. Such methods employ the use of molecules including but not limited to RNA interference, ribozymes, antisense vectors, somatic cell knockouts, intracellular antibodies, etc.

A cell or an animal with defective mismatch repair will become hypermutable. This means that the spontaneous mutation rate of such cells or animals is elevated compared to cells or animals with proficient MMR. The degree of elevation of the spontaneous mutation rate can be at least 2-fold, 5-fold, 10-fold, 20-fold, 50-fold, 100-fold, 200-fold, 500-fold, 1000-fold, or 10,000-fold that of the normal cell or animal. The use of chemical mutagens such as but limited to methane sulfonate, dimethyl sulfonate, O6-methyl benzadine, MNU, ENU, etc. can be used in MMR defective cells to increase the rates an additional 10 to 100 fold that of the MMR deficiency itself.

According to one aspect of the invention, a MMR defective antibody producer cell can be generated by introducing a polynucleotide encoding for a dominant negative form of a MMR protein into a cell. The gene can be any dominant negative allele encoding a protein, which is part of a MMR complex, for example, PMS2, PMS1, MLH1, MLH3, MSH2, MSH3, MSH4, MSH5 or MSH6 (Bocker T, Barusevicius A, Snowden T, Rasio D, Guerrette S, Robbins D, Schmidt C, Burczak J, Croce C M, Copeland T, Kovatich A J, Fishel R. (1999) “hMSH5: a human MutS homologue that forms a novel heterodimer with hMSH4 and is expressed during spermatogenesis” Cancer Res. 59:816-22). The dominant negative allele can be naturally occurring or made in the laboratory. The polynucleotide can be in the form of genomic DNA, cDNA, RNA, or a chemically synthesized polynucleotide.

According to another aspect of the invention a cell line or tissue with a genomic defect in one or a combination of MMR subunits can be used to generate high antibody, Ig or derivative proteins through transfection of genes encoding such proteins whereby a MMR defective cell line producing an antibody, Ig gene, or derivative is generated to yield producer cells. Pools of producer cells are then cloned to identify subclones with enhanced production (referred to as high-titer lines). High titer lines are then made genetically stable by the introduction of a polynucleotide containing wide type gene or DNA fragment that can correct and complement for an endogenous defective MMR gene thereby generating a genetically stable high titer producer line.

The polynucleotide can be cloned into an expression vector containing a constitutively active promoter segment (such as but not limited to CMV, SV40, Elongation Factor, ubiquitin or LTR sequences) or to inducible promoter sequences such as the steroid inducible pIND vector (Invitrogen), where the expression of the dominant negative or wild type MMR gene can be regulated. The polynucleotide can be introduced into the cell by transfection.

According to another aspect of the invention, an immunoglobulin (Ig) gene, a set of Ig genes or a chimeric gene containing whole or parts of an Ig gene can be transfected into MMR deficient cell hosts, the cell is grown and screened for clones producing elevated levels of antibody, Igs or derivatives thereof. MMR defective cells may be of human, primates, mammals, rodent, plant, yeast or of the prokaryotic kingdom. The MMR defective cell encoding the antibody, immunoglobulin or derivative protein with enhanced production may have elevated production through because of increased gene expression, stability, processing and/or secretion. High producer subclones can be genetically analyzed to identify altered gene products whose altered function results in enhanced antibody or Ig production. The method of isolating antibody/Ig enhancer genes may be accomplished using any method known in the art. Candidate genes are validated by altering the expression or function of a candidate gene by introducing via transfection the said gene(s) into the parental line to determine the ability of the altered gene to enhance the production of antibody, immunoglobulin, or derivatives thereof.

Transfection is any process whereby a polynucleotide is introduced into a cell. The process of transfection can be carried out in a living animal, e.g., using a vector for gene therapy, or it can be carried out in vitro, e.g., using a suspension of one or more isolated cells in culture. The cell can be any type of prokaryotic or eukaryotic cell, including, for example, cells isolated from humans or other primates, mammals or other vertebrates, invertebrates, and single celled organisms such as protozoa, yeast, or bacteria.

In general, transfection will be carried out using a suspension of cells, or a single cell, but other methods can also be applied as long as a sufficient fraction of the treated cells or tissue incorporates the polynucleotide so as to allow transfected cells to be grown and utilized. The protein product of the polynucleotide may be transiently or stably expressed in the cell. Techniques for transfection are well known. Available techniques for introducing polynucleotides include but are not limited to electroporation, transduction, cell fusion, microinjection, the use of calcium chloride, and packaging of the polynucleotide together with lipid for fusion with the cells of interest. Once a cell has been transfected with the candidate gene, the cell can be grown and reproduced in culture. If the transfection is stable, such that the gene is expressed at a consistent level for many cell generations, then a cell line results.

An isolated cell is a cell obtained from a tissue of plants or animals by mechanically separating out individual cells and transferring them to a suitable cell culture medium, either with or without pretreatment of the tissue with enzymes, e.g., collagenase or trypsin. Such isolated cells are typically cultured in the absence of other types of cells. Cells selected for the introduction of a candidate Antibody/Ig Enhancer Gene may be derived from a eukaryotic or prokaryotic organism in the form of a primary cell culture or an immortalized cell line, or may be derived from suspensions of single-celled organisms.

Mutant genes in antibody over-producing cells can be detected by analyzing for alterations in the genotype of the cells or animals, for example by examining the sequence of genomic DNA, cDNA, messenger RNA, or amino acids associated with the gene of interest. Mutations can also be detected by screening for the production of antibody or Ig titers. A mutant polypeptide can be detected by identifying alterations in electrophoretic mobility, spectroscopic properties, or other physical or structural characteristics of a protein encoded by a mutant gene. One can also screen for altered function of the protein in situ, in isolated form, or in model systems. One can screen for alteration of any property of the cell or animal associated with the function of the gene of interest, such as but not limited to Ig secretion.

Another aspect of the invention is the composition of matter and methods of use whereby two genes, alpha-1-anti-trypsin (AAT) (SEQ ID NO:1) and endothelial monocyte-activating polypeptide I (EMAP) (SEQ ID NO:2) were identified to be significantly suppressed in high titer antibody producer cells. Functional studies have demonstrated that the decreased expression of these genes in parental cell lines using antisense technology can lead to enhanced antibody production. Conversely, the over-expression of these genes in high producer lines that lack robust expression of either the AAT and/or EMAP protein or pathway can suppress antibody expression demonstrating the utility of these genes for regulating antibody production from producer cells.

Another aspect of the invention employs the use of chemical inhibitors (such as those described in WO 02/054856) that block the biological pathway of the AAT and/or EMAP gene products that leads to increased antibody production demonstrating the use of small molecules of the genes pathway as a method for enhancing antibody/Ig gene production.

Yet another aspect of the invention is the regulation of the AAT and/or EMAP protein by biological or chemical agents for the use in modulating their biological pathway for controlling immunoglobulin gene expression in immunological-associated disease states such as allergy and inflammation.

In some embodiments, the invention comprises a host cell for the expression of antibody molecules or fragments thereof comprising a defect in the monocyte-activating polypeptide I gene such that expression of monocyte-activating polypeptide I is inhibited. These cells may have a defect such as a deletion of monocyte-activating polypeptide I and/or alpha-1-antitrypsin, or a frameshift mutation in one or both of these genes. Alternatively, the host cell may comprise an expression vector comprising an antisense transcript of the monocyte-activating polypeptide I gene and/or alpha-1-antitrypsin gene whereby expression of said antisense transcript suppresses the expression of the gene. In other embodiments, the host cell may comprise a ribozyme that disrupts expression of the monocyte-activating polypeptide I gene or an intracellular neutralizing antibody or antibodies against the monocyte-activating polypeptide I protein and/or alpha-1-antitrypsin protein whereby the antibody or antibodies suppress the activity of the protein(s).

The host cells are useful for expressing antibody molecules in high titer and thus may further comprise polynucleotides encoding fully human antibodies, human antibody homologs, humanized antibody homologs, chimeric antibody homologs, Fab, Fab′, F(ab′)₂ and F(v) antibody fragments, single chain antibodies, and monomers or dimers of antibody heavy or light chains or mixtures thereof.

The cells of the invention may include mammalian cells, bacterial cells, plant cells, and yeast cells.

The method of the invention may also comprise restabilizing the genome of the cells of the invention that are expressing antibodies in high titers. This can be achieved by the use of inducible vectors whereby dominant negative MMR genes are cloned into such vectors, introduced into Ab producing cells and the cells are cultured in the presence of inducer molecules and/or conditions. Inducible vectors include but are not limited to chemical regulated promoters such as the steroid inducible MMTV, tetracycline regulated promoters, temperature sensitive MMR gene alleles, and temperature sensitive promoters. This may also be accomplished by procedures to remove the vectors containing the dominant negative alleles from the selected cells. Such procedures for removing plasmids from cells are well-known in the art.

For further information on the background of the invention the following references may be consulted, each of which is incorporated herein by reference in its entirety:
1. Glaser, V. (1996) Can ReoPro repolish tarnished monoclonal therapeutics? Nat. Biotechol. 14:1216-1217.
2. Weiner, L. M. (1999) Monoclonal antibody therapy of cancer. Semin. Oncol. 26:43-51.
3. Saez-Llorens, X. E. et al. (1998) Safety and pharmacokinetics of an intramuscular humanized monoclonal antibody to respiratory syncytial virus in premature infants and infants with bronchopulmonary dysplasia. Pediat. Infect. Dis. J. 17:787-791.
4. Shield, C. F. et al. (1996) A cost-effective analysis of OKT3 induction therapy in cadaveric kidney transplantation. Am. J. Kidney Dis. 27:855-864.
5. Khazaeli, M. B. et al. (1994) Human immune response to monoclonal antibodies. J. Immunother. 15:42-52.
6. Emery, S. C. and W. J. Harris “Strategies for humanizing antibodies” In: Antibody Engineering C. A. K. Borrebaeck (Ed.) Oxford University Press, N.Y. 1995, pp. 159-183.
7. U.S. Pat. No. 5,530,101 to Queen and Selick.
8. Reff, M. E. (1993) High-level production of recombinant immunoglobulins in mammalian cells. Curr. Opin. Biotechnol. 4:573-576.
9. Neuberger, M. and M. Gruggermann, (1997) Monoclonal antibodies. Mice perform a human repertoire. Nature 386:25-26.
10. Fiedler, U. and U. Conrad (1995) High-level production and long-term storage of engineered antibodies in transgenic tobacco seeds. Bio/Technology 13:1090-1093.
11. Baker S. M. et al. (1995) Male defective in the DNA mismatch repair gene PMS2 exhibit abnormal chromosome synapsis in meiosis. Cell 82:309-319.
12. Bronner, C. E. et al. (1994) Mutation in the DNA mismatch repair gene homologue hMLH1 is associated with hereditary non-polyposis colon cancer. Nature 368:258-261.
13. de Wind N. et al. (1995) Inactivation of the mouse Msh2 gene results in mismatch repair deficiency, methylation tolerance, hyperrecombination, and predisposition to cancer. Cell 82:321-300.
14. Drummond, J. T. et al. (1995) Isolation of an hMSH2-p160 heterodimer that restores mismatch repair to tumor cells. Science 268:1909-1912.
15. Modrich, P. (1994) Mismatch repair, genetic stability, and cancer. Science 266: 1959-1960.
16. Nicolaides, N. C. et al. (1998) A Naturally Occurring hPMS2 Mutation Can Confer a Dominant Negative Mutator Phenotype. Mol. Cell. Biol. 18:1635-1641.
17. Prolla, T. A. et al. (1994) MLH1, PMS1, and MSH2 Interaction during the initiation of DNA mismatch repair in yeast. Science 264:1091-1093.
18. Strand, M. et al. (1993) Destabilization of tracts of simple repetitive DNA in yeast by mutations affecting DNA mismatch repair. Nature 365:274-276.
19. Su, S. S., R. S. Lahue, K. G. Au, and P. Modrich (1988) Mispair specificity of methyl directed DNA mismatch corrections in vitro. J. Biol. Chem. 263:6829-6835.
20. Parsons, R. et al. (1993) Hypermutability and mismatch repair deficiency in RER⁺ tumor cells. Cell 75:1227-1236.
21. Papadopoulos, N. et al. (1993) Mutation of a mutL homolog is associated with hereditary colon cancer. Science 263:1625-1629.
22. Perucho, M. (1996) Cancer of the microsatellite mutator phenotype. Biol. Chem. 377:675-684.
23. Nicolaides N. C., K. W. Kinzler, and B. Vogelstein (1995) Analysis of the 5′ region of PMS2 reveals heterogenous transcripts and a novel overlapping gene. Genomics 29:329-334.
24. Nicolaides, N. C. et al. (1995) Genomic organization of the human PMS2 gene family. Genomics 30:195-206.
25. Palombo, F. et al. (1994) Mismatch repair and cancer. Nature 36:417.
26. Eshleman J. R. and S. D. Markowitz (1996) Mismatch repair defects in human carcinogenesis. Hum. Mol. Genet. 5:1489-494.
27. Liu, T. et al. (2000) Microsatellite instability as a predictor of a mutation in a DNA mismatch repair gene in familial colorectal cancer. Genes Chromosomes Cancer 27:17-25.
28. Nicolaides, N. C. et al. (1992) The Jun family members, c-JUN and JUND, transactivate the human c-myb promoter via an Ap1 like element. J. Biol. Chem. 267: 19665-19672.
29. Shields, R. L. et al. (1995) Anti-IgE monoclonal antibodies that inhibit allergen-specific histamine release. Int. Arch. Allergy Immunol. 107:412-413.
30. Frigerio L. et al. (2000) Assembly, secretion, and vacuolar delivery of a hybrid immunoglobulin in plants. Plant Physiol. 123:1483-1494.
31. Bignami M, (2000) Unmasking a killer: DNA O(6)-methylguanine and the cytotoxicity of methylating agents. Mutat. Res. 462:71-82.
32. Drummond, J. T. et al. (1996) Cisplatin and adriamycin resistance are associated with MutLa and mismatch repair deficiency in an ovarian tumor cell line. J. Biol. Chem. 271:9645-19648.
33. Galio, L. et al. (1999) ATP hydrolysis-dependent formation of a dynamic ternary nucleoprotein complex with MutS and MutL. Nucl. Acids Res. 27:2325-23231.

The above disclosure generally describes the present invention. A more complete understanding can be obtained by reference to the following specific examples which are provided herein for purposes of illustration only, and are not intended to limit the scope of the invention.

### EXAMPLE 1

### Generation of Mismatch Repair Defective Cells for Generating Enhanced Antibody/Immunoglobulin Producer Lines

Expression of a dominant negative allele in an otherwise MMR proficient cell can render these host cells MMR deficient. The creation of MMR deficient cells can lead to the generation of genetic alterations throughout the entire genome of a host organism's offspring, yielding a population of genetically altered offspring or siblings that may produce biochemicals with altered properties.

It has been discovered that MMR defective cells are useful for creating high-titer antibody-producer cells, including but not limited to rodent hybridomas, human hybridomas, surrogate rodent cells producing human immunoglobulin gene products, surrogate human cells expressing immunoglobulin genes, eukaryotic cells producing single chain antibodies, and prokaryotic cells producing mammalian immunoglobulin genes and/or chimeric immunoglobulin molecules such as those contained within single-chain antibodies. The cell expression systems described above that are used to produce antibodies are well known by those skilled in the art of antibody therapeutics.

To demonstrate the ability to create MMR defective surrogate cell lines and hybridomas using dominant negative alleles of MMR genes, we first transfected a mouse hybridoma cell line (cell line referred to H6) that is known to produce and antibody directed against the IgE protein with an expression vector containing the previously published dominant negative PMS2 mutant referred herein as PMS134 (cell line referred to as H34), or empty vector (cell line referred to as H6vec) or the rodent Chinese hamster ovary (CHO) line (parental referred to as CHO-P and the dominant negative MMR cell referred to as CHO-34). The results showed that the PMS134 mutant exerts a robust dominant negative effect, resulting in biochemical and genetic manifestations of MMR deficiency as determined by the ability to enhance microsatellite instability of a reporter gene (not shown), which is a hallmark of MMR deficiency as well as increased point mutations that lead to the accumulation of mutations in metabolic genes such as the hypoxanthine phosphoribosyltransferase (HPRT) gene leading to subclones that can grow under selective conditions using methods known by those skilled in the art (Qian Y, Yu Y, Cheng X, Luo J, Xie H, Shen B. Molecular events after antisense inhibition of hMSH2 in a HeLa cell line. Mutat Res 1998 418:61-71). As shown in TABLE 1, CHO cells were preselected to remove spontaneous HPRT mutants that have accumulated over the course of standard propagation and then screened for defected HPRT to determine rate of mutagenesis. Briefly, CHO-P and CHO-34 cells were then grown for 40 doublings and one hundred thousand cells were selected for mutations at the HPRT locus using 6.7 ug/ml of 6-thioguanine in growth medium and scored for resistant colonies at day 10. Colony numbers are based out of one million cells screened.

TABLE 1
HPRT mutations in parental and mismatch repair defective CHO cells

| CELL LINE | CELLS SCREENED | HPRT MUTANTS |
|---|---|---|
| CHO-P | 1,000,000 | 1 +/− 1.7 |
| CHO-34 | 1,000,000 | 62 +/− 10 |

MMR defective cells are now ready to be transfected with immunoglobulin genes and screened to identify subclones with enhanced titer yields or in the case cells already containing expressed immunoglobulin light and heavy chains such as hybridomas, be expanded and screened directly for high titer production lines.

### EXAMPLE 2

### Screening of Hybridoma Clones with Increased Immunoglobulin Production for Gene Discovery

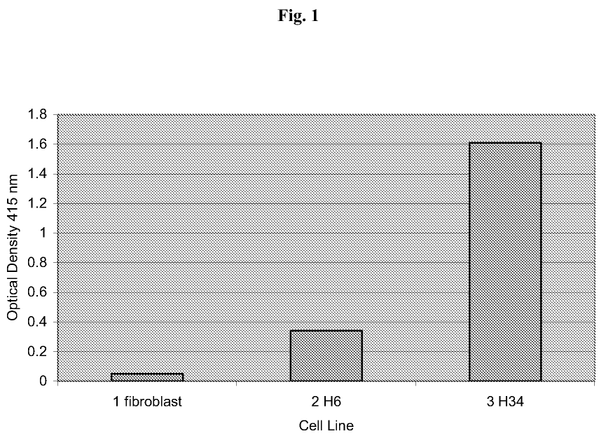

An application of the methods presented within this document is the use of MMR deficient hybridomas or MMR defective surrogate cells that can be transfected with immunoglobulin genes such as CHO (see Example 1, Table 1), BHK, NSO, SPO-2, etc., to generate high titer. An illustration of this application is demonstrated within this example whereby the H34 hybridoma, in which a murine MMR-defective cell line producing a mouse IgG monoclonal antibody was grown for 20 generations and clones were isolated in 96-well plates and screened for antibody production. The screening procedure to identify clones that produce high levels of antibody, which is presumed to be due to an alteration within the genome of the host cell line is an assay that employs the use of a plate Enzyme Linked Immunosorbant Assay (ELISA) to screen for clones that produce enhanced antibody titers. 96-well plates containing single cells from H6 parental or H34 pools were grown for 9 days in growth medium (RPMI 1640 plus 10% fetal bovine serum) plus 0.5 mg/ml G418 to ensure clones retain the dominant negative MMR gene expression vector. After 9 days, plates were screened using an anti-Ig ELISA, whereby a 96 well plate is coated with 50 uls of conditioned supernatant from independent clones for 4 hours at 4° C. Plates were washed 3 times in calcium and magnesium free phosphate buffered saline solution (PBS^{-/-}) and blocked in 100 uls of PBS^{-/-} containing 5% dry milk for 1 hour at room temperature. Plates were then washed 3 times with PBS^{-/-} and incubated for 1 hour at room temperature with 50 uls of a PBS^{-/-} solution containing 1:3000 dilution of a sheep anti-mouse horse radish peroxidase (HRP) conjugated secondary antibody. Plates were then washed 3 times with PBS^{-/-} and incubated with 50 uls of TMB-HRP substrate (BioRad) for 15 minutes at room temperature to detect amount of antibody produced by each clone. Reactions were stopped by adding 50 uls of 500 mM sodium bicarbonate and analyzed by OD at 450 nm using a BioRad plate reader. Clones exhibiting an enhanced signal over background cells (H6 control cells) were then isolated and expanded into 10 ml cultures for additional characterization and confirmation of ELISA data in triplicate experiments. Clones that produce an increased ELISA signal and have increased antibody levels were then further analyzed for variants that over-express and/or over-secrete antibodies as described in Example 4. Analysis of five 96-well plates each from H6 or H34 cells have found that a significant number of clones with a higher Optimal Density (OD) value is observed in the MMR-defective H34 cells as compared to the H6 controls. FIG. 1 shows a representative example of H34 clones producing enhanced levels of antibody. FIG. 1 provides primary data from the analysis of 96 wells of fibroblast conditioned medium as negative control (lane 1), H6 (lane 2) or H34 (lane 3) cultures which shows clones from the H34 plate to have a higher OD reading due to genetic alteration of a cell host that leads to over-production/secretion of the antibody molecule.

Clones that produce higher OD values due to enhanced antibody production are sequenced to confirm that mutations have not occurred within the light or heavy chain cDNA. Briefly, 100,000 cells are harvested and extracted for RNA using the Trizol method as described above. RNAs are reverse transcribed using Superscript II as suggested by the manufacturer (Life Technology) and PCR amplified for the full-length light and heavy chains.

These data demonstrate the ability to generate hypermutable hybridomas, or other Ig producing host cells that can be grown and selected, to identify subclones with enhanced antibody/Ig production due to putative structural alterations that have occurred within genome of the host cell that are involved in enhancing antibody production through increased gene expression, protein stability, processing or secretion. Clones can also be further expanded for subsequent rounds of in vivo mutations and can be screened yet higher titer clones due to the accumulation of mutations within additional gene(s) involved in enhancing production. Moreover, the use of chemical mutagens to produce additional genetic mutations in cells or whole organisms can enhance the mutation spectrum in MMR defective cells as compared to “normal” cells. The use of chemical mutagens such as MNU in MMR defective organisms is much more tolerable yielding to a 10 to 100 fold increase in genetic mutation over MMR deficiency alone (Bignami M, (2000) Unmasking a killer: DNA O(6)-methylguanine and the cytotoxicity of methylating agents. Mutat. Res. 462:71-82). This strategy allows for the use of chemical mutagens to be used in MMR-defective antibody producing cells as a method for increasing additional mutations within the host's genome that may yield even higher titer producer strains.

### EXAMPLE 3

### Use of High Titer Antibody/Immunoglobulin Producer Cells to Identify Genes Involved in Enhancing Antibody or Secreted Protein Production

High titer subclones of hybridomas or surrogate antibody/immunoglobulin gene producer cells can be used as a source for gene target discovery to identify genes involved in enhancing antibody titers for use in developing universal high titer production strains for manufacturing and/or for identifying target genes and pathways involved in up or down regulating immunoglobulin production for therapeutic development of immunological disorders such as allergy and inflammation. A benefit of using MMR derived mutants as compared to chemical or ionizing mutagenesis is the observation that cells that are defective for MMR have increased mutation rates yet retain their intact chromosomal profile (Lindor N M, Jalal S M, Van DeWalker T J, Cunningham J M, Dahl R J, Thibodeau S N. Search for chromosome instability in lymphocytes with germ-line mutations in DNA mismatch repair genes. Cancer Genet Cytogenet 1998 104:48-51). This feature makes genomic analysis of variants more straightforward because of the decreased background noise that is associated with chemical and radiomutagenesis whereby whole increases and decreases of chromosomal content are associated with the mutagenesis process.

To identify variant gene(s) in high-titer antibody/Ig or derivative producer strains, DNA, RNA and proteins are compared for altered expression or structural patterns used by those skilled in the art. Such techniques employ single polynucleotide analysis (also referred to SNP analysis) which can recognize single nucleotide changes in transcripts of genomic or reverse transcribed RNA templates; microarray or subtractive analysis which can recognize differences in RNA expression profiles; or proteomic analysis which can identify differences in protein profiles between parental and variant lines. Once candidate DNA, transcript or proteins are identified candidates are validated for their role in over-production by: 1.) steady state RNA and/or protein levels and 2.) alteration (over-expression, suppression, and/or introduction of mutant gene) of candidate gene in parental cell line to demonstrate the ability of said candidate gene(s) to recapitulate the over-expression phenotype.

One method for detection of expression patterns among various alternatives, differential expression analysis of H6 parental and H34 high-titer lines, was performed using microarray methods. Analysis of steady state transcripts identified two genes (SEQ ID NO:1 and SEQ ID NO:2) whose expression is suppressed in the high titer H34 cell line. Expression analysis of both genes was carried out using reverse transcriptase coupled polymerase chain reaction (RT-PCR). The putative genes encoded for the murine alpha-1-anti-trypsin (referred to as AAT) (SEQ ID NO:1, accession number 100556; U.S. Pat. No. 4,732,973; U.S. Pat. No. 4,732,973-A 2) and the murine endothelial monocyte-activating polypeptide I (referred to as EMAPI) (SEQ ID NO:2 accession number U41341). RNAs were reverse transcribed as described (Nicolaides, N. C. et al. (1995) Genomic organization of the human PMS2 gene family. Genomics 30:195-206). Sense and antisense primers were generated that can specifically amplify the AAT cDNA to yield a 540 bp product and EMAPI cDNA to yield a 272 bp product as listed below while the dihydrofolate reductase (DHFR) cDNA was used as a control to monitor RNA integrity and reaction performance using primers as previously described (Nicolaides, N. C., et. al. Interleukin 9: A candidate gene for asthma. 1997 Proc. Natl. Acad. Sci USA 94:13175-13180).

Primers murine AAT and EMAP expression analysis

SEQ ID NO:3

| | |
|---|---|
| AAT sense | 5′-ttgaagaagccattcgatcc-3′ |

SEQ ID NO:4

| | |
|---|---|
| AAT antisense | 5′-tgaaaaggaaagggtggtcg-3′ |

SEQ ID NO:5

| | |
|---|---|
| EMAPI sense | 5′-atgcctacagagactgagag-3′ |

SEQ ID NO:6

| | |
|---|---|
| EMAPI antisense | 5′-gattcgcttctgggaagtttgg-3′ |

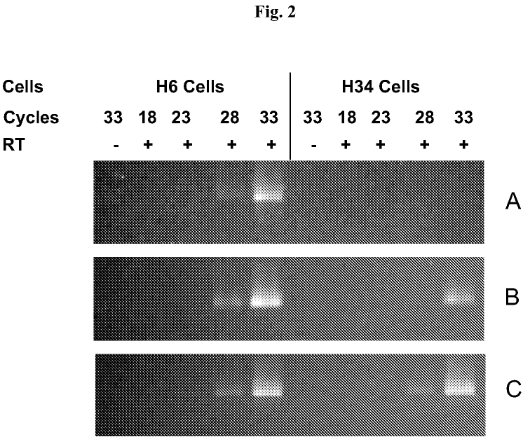

PCR reactions were carried out at 95° C. for 30 sec, 58° C. for 1 min, 72° C. for 1 min for 18 to 33 cycles to measure expression over a linear range. FIG. 2 demonstrates a representative profile of steady state expression for the AAT and EMAPI genes in the H6 parental and H34 over-producer strain. As shown, a significant loss of expression was observed in the H34 over producer line for both AAT and EMAPI as compared to the parental control. DHFR expression levels were similar for both samples indicating intact RNA and equal loadings for both samples. These data suggest a roll for AAT and EMAPI in regulating antibody production in mammalian cells.

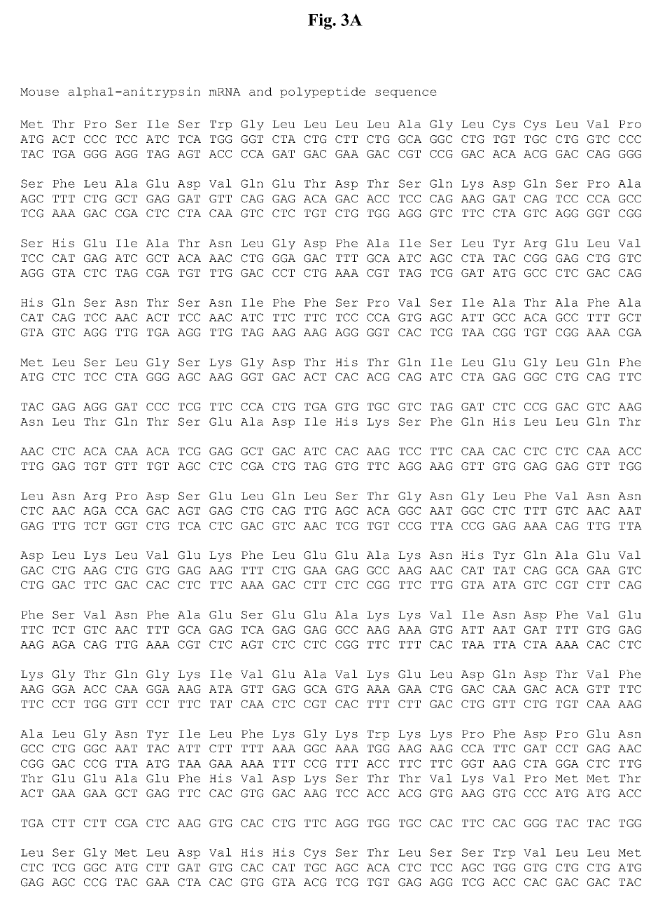

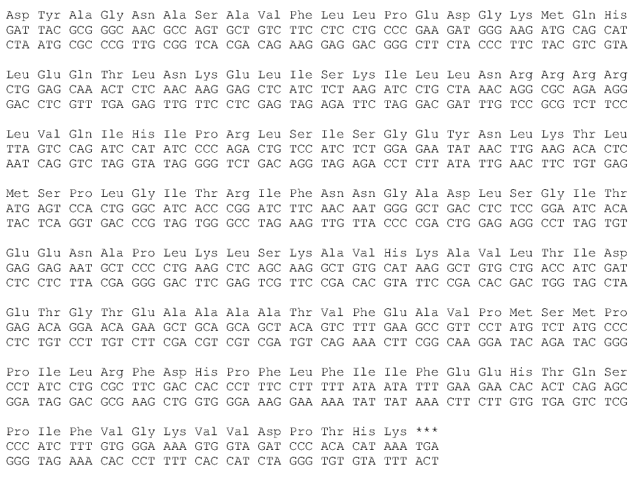

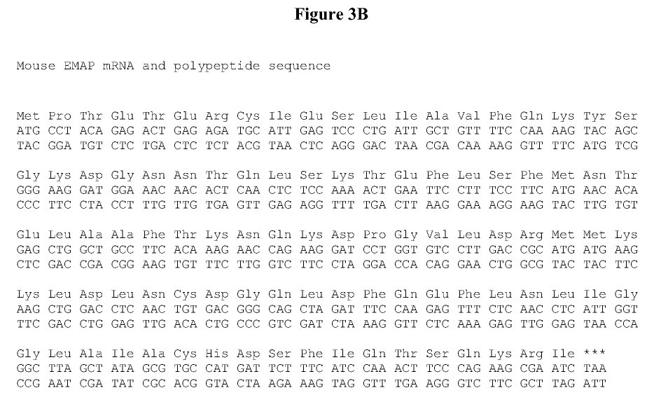

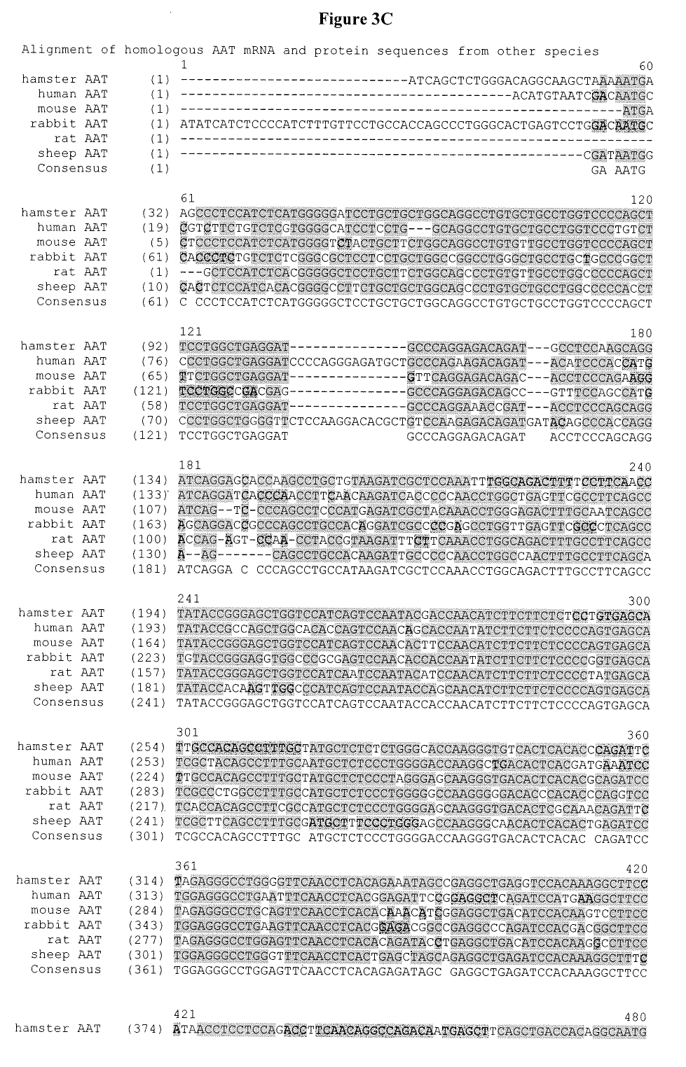

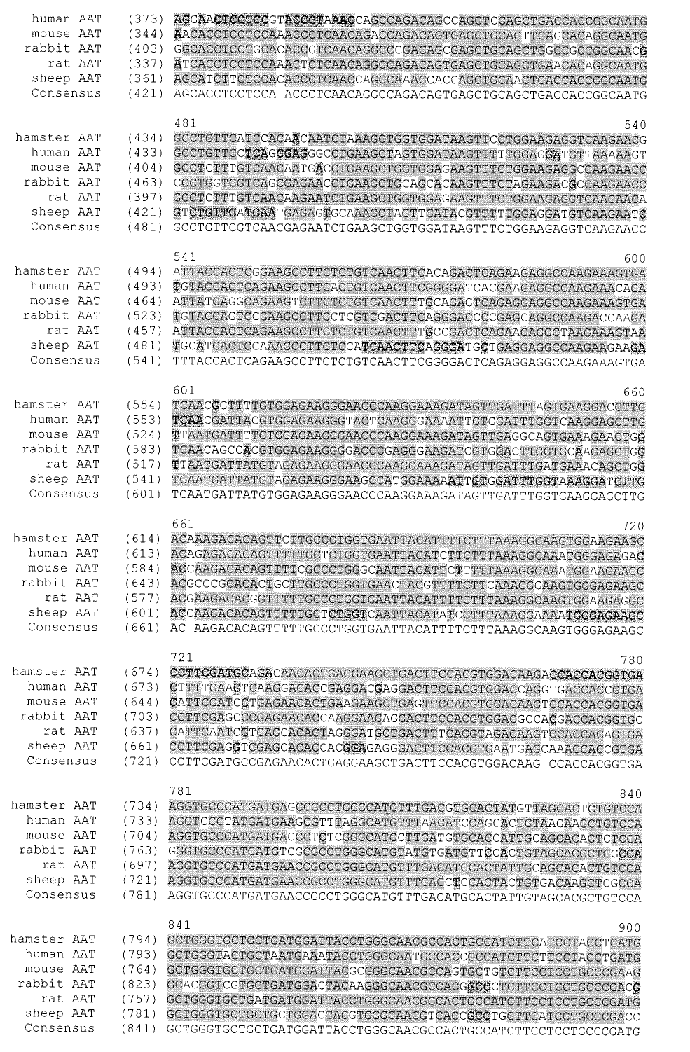

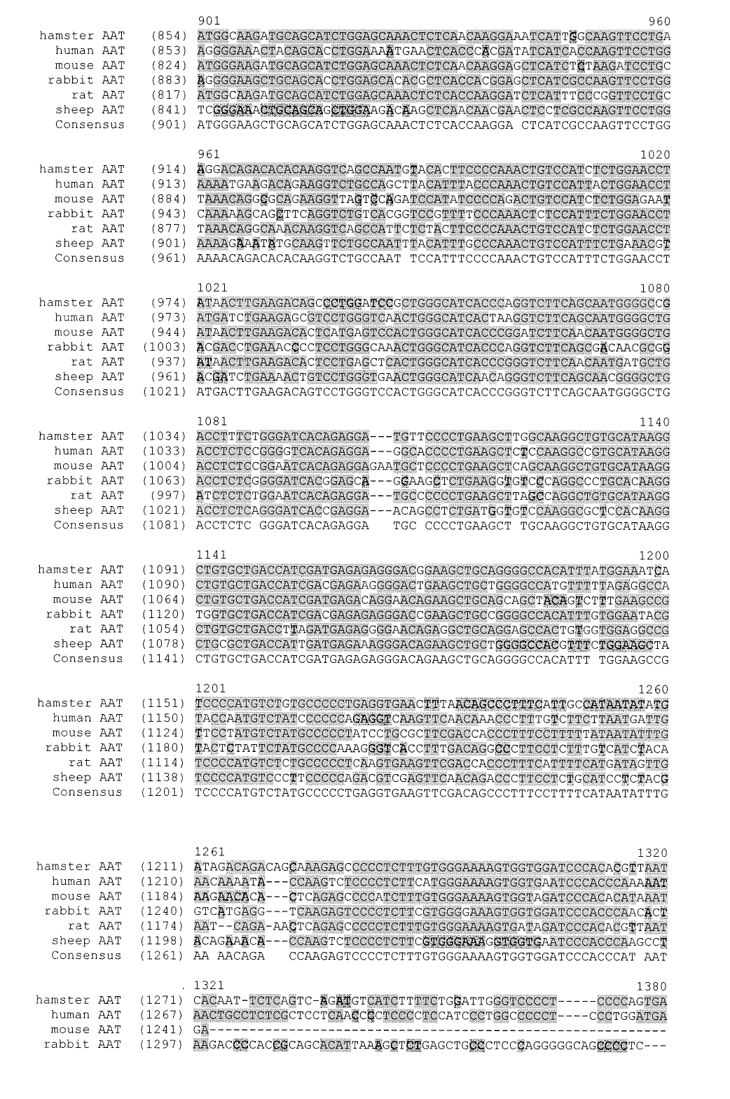

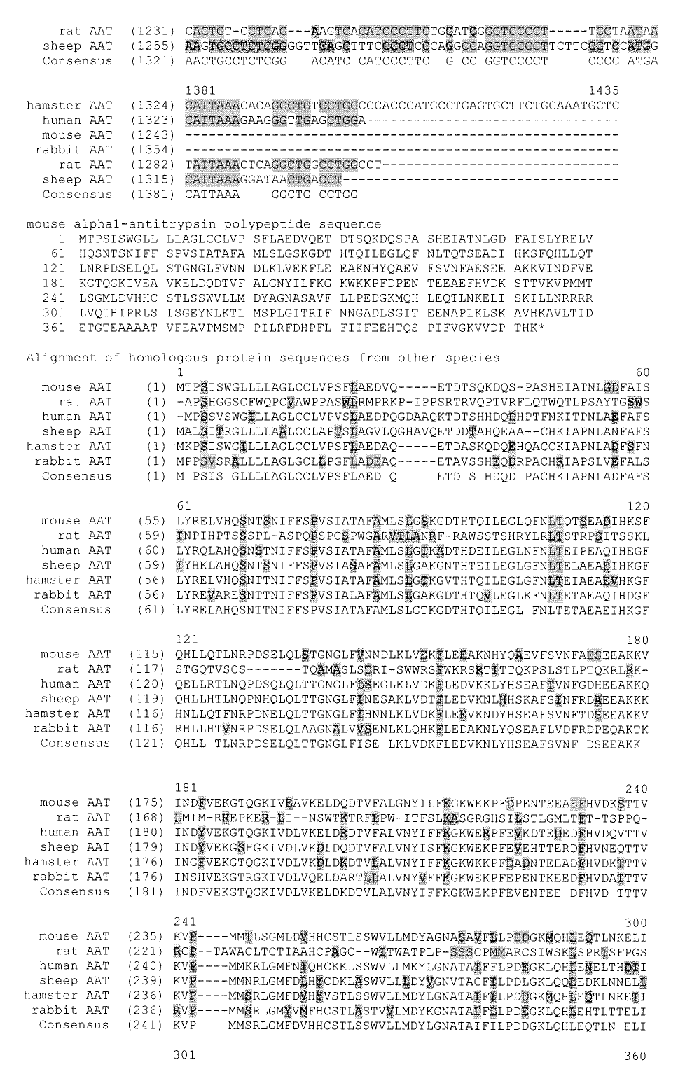

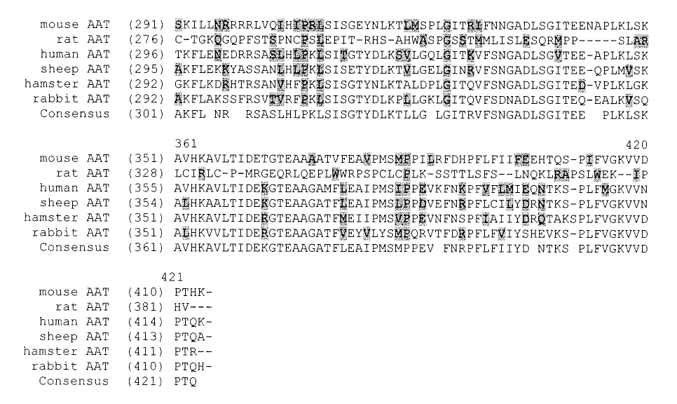

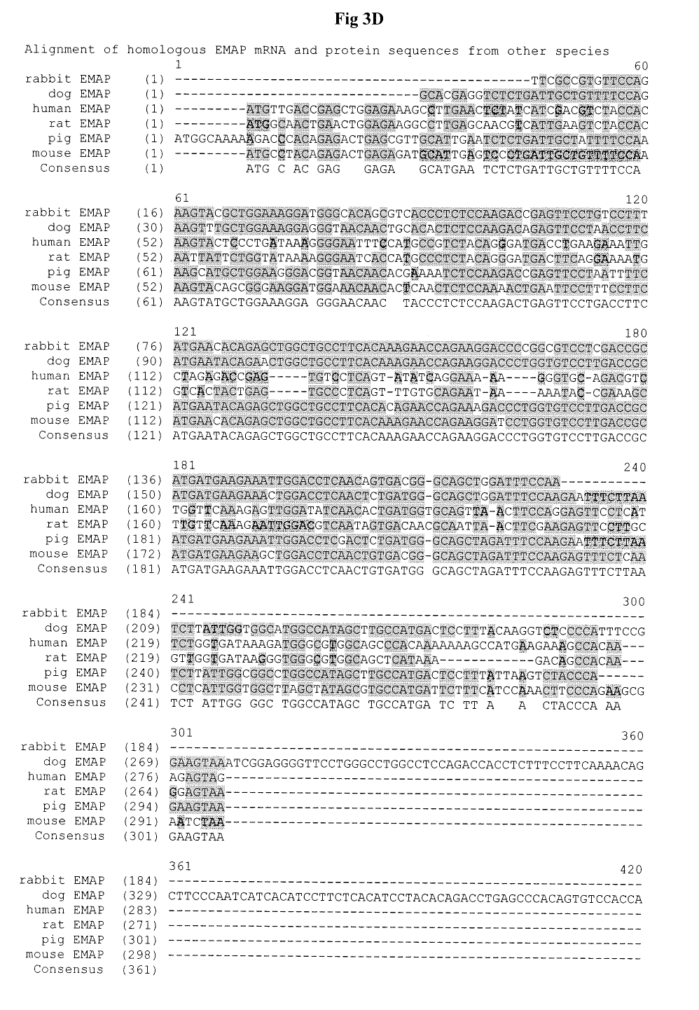

To confirm that these proteins or lack thereof are involved in regulating antibody production, we have isolated the full-length cDNAs for each gene to be cloned into the sense and/or antisense direction of a mammalian expression vector. FIG. 3 shows the isolated cDNA and predicted encoded polypeptide for the murine alpha-1-anti-trypsin (FIG. 3A) and the murine endothelial monocyte-activating polypeptide I (FIG. 3B). Because of their possible role in regulating antibody or immunoglobulin production in mammalian systems we performed a blast search and identified AAT homologs from hamster (SEQ ID NO:7), human (SEQ ID NO:8), rabbit (SEQ ID NO:9), rat (SEQ ID NO:10), and sheep (SEQ ID NO:11) (FIG. 3C) and EMAPI homologs from rabbit (SEQ ID NO:12), dog (SEQ ID NO:13), human (SEQ ID NO:14), rat (SEQ ID NO:15), and pig (SEQ ID NO:16) (FIG. 3D) that can be of use for enhancing antibody/immunoglobulin production from cells derived from any of these respective species.

To directly confirm the involvement of AAT and/or EMAPI in regulating antibody production, we generated mammalian expression vectors to produce sense and anti-sense RNAs in parental H6 or over-producer H34 cell lines. If suppression of either or both genes are involved in antibody production, then we would expect enhanced expression in parental lines when treated with antisense vectors that can suppress the AAT and/or EMAP expression levels. Conversely, we should expect to suppress antibody production levels in over producer H34 cells upon reestablished expression of either or both genes. Expression vectors were generated in pUC-based vectors containing the constitutively active elongation factor-1 promoter followed by the SV40 polyA signal. In addition, AAT vectors had a hygromycin selectable marker while EMAP vectors had neomycin selectable markers to allow for double transfection/selection for each vector.

Combinations of antisense AAT and EMAPI vectors were transfected into the parental H6 cell using polyliposomes as suggested by the manufacturer (Gibco/BRL) and stable lines were selected for using 0.5 mg/ml of hygromycinB and the neomycin analog G418. After two weeks of selection, stable clones were derived, expanded and analyzed for sense or antisense gene expression using northern and RT-PCR analysis. Positive clones expressing each vector were then expanded and tested for antibody production using ELISA analysis as described in EXAMPLE 2. Briefly, stable lines or controls were plated at 50,000 cells in 0.2 mls of growth medium per well in triplicates in 96 well microtiter dishes. Cells were incubated at 37° C. in 5% CO₂ for 5 days and 50 uls of supernatant was assayed for antibody production. H6 cells expressing the antisense AAT and EMAPI produced enhanced levels of antibody in contrast to parental control or H6 cells expressing sense AAT and EMAP1. Conversely, H34 cells (expressing enhanced antibody levels) expressing sense AAT and EMAPI were found to have suppressed antibody production in contrast to H6 parental expressing sense AAT and EMAPI (TABLE 2). These data demonstrate the involvement of AAT and EMAPI in regulating antibody production. Moreover, these data teach us of the use of modulating the expression or function of each of these genes for enhancing or suppressing antibody production for use in developing high titer protein manufacturing strains as well as their use in treating immunological disorders involving hyper or hypo immunoglobulin production.

TABLE 2
Antisense suppression of AAT and EMAPI results in enhanced
antibody production in H6 cells. Restored AAT and EMAPI
expression in H34 over-producer cells results in suppressed
antibody production.

| Cell Line | Antibody (ug/ml) |
|---|---|
| H6 | 13134 +/− 992 |
| H6 AS AAT/EMAP | 29138 +/− 880 |
| H34 | 38452 +/− 1045 |
| H34 sense AAT/EMAP | 14421 +/− 726 |

### EXAMPLE 5

### Use of Small Molecules Targeted Against the Alpha-1-Anti-Trypsin Pathway for Modulating Antibody Production

The finding as taught by this application that increasing protease activity via suppressing a natural inhibitor such as alpha-1-antitrypsin may lead to increased antibody production suggests that molecules that alter protease activity may be useful for generating enhanced or suppressed immunoglobulin production from producer lines for use in increasing productivity for manufacturing and/or for use in immunoglobulin regulation of immunological disease. To test the hypothesis, we first used a small molecule protease inhibitor called 4-(2-aminoethyl)-benzenesulfonyl floride (AEBSF), which is a potent trypsin inhibitor (Lawson W B, Valenty V B, Wos J D, Lobo A P. Studies on the inhibition of human thrombin: effects of plasma and plasma constituents. Folia Haematol Int Mag Klin Morphol Blutforsch 1982 109:52-60). Briefly, H34 cells were incubated for 1-3 days in the presence of 4 mM AEBSF in 96 well plates and supernatants were tested for antibody production by ELISA. As shown in TABLE 3, H34 cells had a significant suppression of antibody production (0.031 ug/ml) as compared to untreated H34 cells (4.3 ug/ml).

Next, we tested the ability of antiserum directed against AAT (see Example 6 for generation of antiserum) to effect antibody production from H6 lines. If increased protease activity is associated with increased production, then sequestration of a protease inhibitor may increase antibody production. As shown in TABLE 3, H6 parental cells grown in the presence of anti-AAT had increased antibody production (2.6 ug/ml) as compared to H6 cells exposed to preimmune serum (1.6 ug/ml). These data imply the use of protease activators or inhibitors to modulate antibody production for manufacturing as well as to treat immune disorders associated with hyper or hypo immunoglobulin production.

TABLE 3
Antibody production from hybridomas incubated with protease
inhibitors or inhibitors of natural proteases.

| | | ANTIBODY | ANTIBODY |
|---|---|---|---|
| | | PRODUCTION | PRODUCTION |
| CELL LINE | TREATMENT | UNTREATED | TREATED |
| H34 AEBSF | AEBSF | 4.3 ug/ml | 0.031 ug/ml |
| H6 | PREIMMUNE | — | 1.6 ug/ml |
| H6 | ANTI-ALPHA-1- | — | 2.6 ug/ml |
| | ANTITRYPSIN | | |

### EXAMPLE 6

### Use of Antibodies to Alpha-1-Antitrypsin and/or Endothelial Monocyte-Activating Polypeptide I for Screening of Cell Clones for Enhanced or Suppressed Immunoglobulin Production

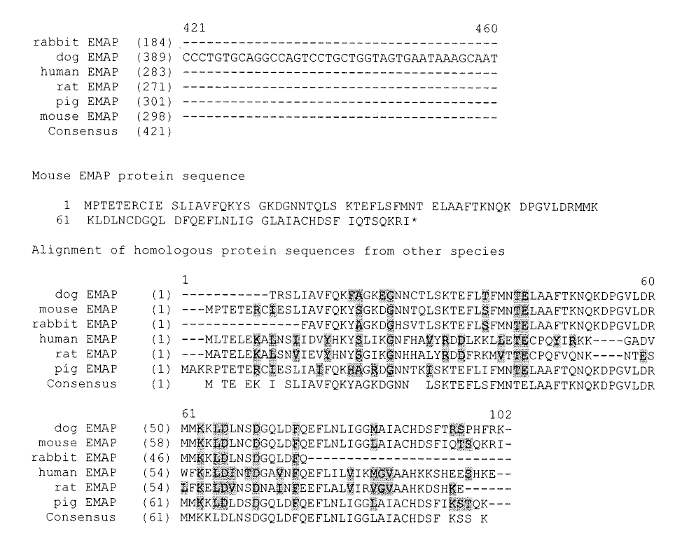

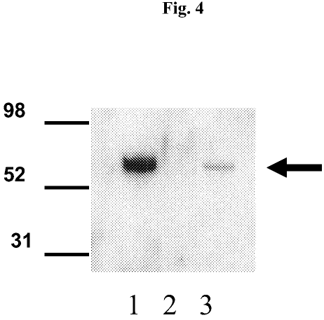

The associated lack of AAT and EMAPI expression with enhanced antibody production from producer strains is useful for screening for high antibody production strains. To demonstrate this utility, we generated monoclonal antiserum against the murine AAT and murine EMAPI protein using polypeptides (SEQ ID NO:17-AAT:(C)QSPIFVGKVVDPTHK and SEQ ID NO:18-EMAPI: (C)IACHDSFIQTSQKRI) derived from their respective translated proteins using methods used by those skilled in the art. We next tested the ability of these antisera to detect protein in the conditioned medium of H6 and H34 cells since both proteins are secreted polypeptides. Briefly, conditioned medium from 10,000 cells were prepared for western blot analysis to assay for steady state protein levels (FIG. 4). Briefly, cells were pelleted by centrifugation and 100 uls of conditioned supernatant were resuspended in 300 ul of SDS lysis buffer (60 mM Tris, pH 6.8, 2% SDS, 10% glycerol, 0.1 M 2-mercaptoethanol, 0.001% bromophenol blue) and boiled for 5 minutes. Proteins were separated by electrophoresis on 4-12% NuPAGE gels (for analysis of Ig heavy chain. Gels were electroblotted onto Immobilon-P (Millipore) in 48 mM Tris base, 40 mM glycine, 0.0375% SDS, 20% methanol and blocked at room temperature for 1 hour in Tris-buffered saline (TBS) plus 0.05% Tween-20 and 5% condensed milk. Filters were probed with a 1:1000 dilution of mouse anti-AAT or mouse anti-EMAP antiserum in TBS buffer for 1 hour at room temperature. Blots were washed three times in TBS buffer alone and probed with a 1:10000 dilution of sheep anti-mouse horseradish peroxidase conjugated monoclonal antibody in TBS buffer and detected by chemiluminescence using Supersignal substrate (Pierce). Experiments were repeated in duplicates to ensure reproducibility. FIG. 4 shows a representative analysis where low producer H6 parental cells (Lane 1) had robust, steady-state AAT protein levels while no expression was observed in H34 over producer cells (Lane 2). These data suggest a method for screening of cell lines for expression of AAT or EMAP to identify high-titer producer strains that can be used to manufacture high levels of antibody or recombinant polypeptides.

The results described above lead to several conclusions. First, the use of mismatch repair defective cells can be used to generate high titer antibody producer cells. Secondly, the generation of high titer producer lines using this method can be used to identify gene(s) involved in increased antibody production. Finally, the methods that can modulate the expression and/or biological activity of the alpha-1-antitrypsin and/or endothelial monocyte-activating polypeptide I can be used to up or down-regulate antibody/immunoglobulin protein production in cells for manufacturing and/or the treatment of immunological-based disorders involving hyper or hype immunoglobulin production (Shields, R. L., et al. (1995) Anti-IgE monoclonal antibodies that inhibit allergen-specific histamine release. Int. Arch Allergy Immunol. 107:412-413).

What is claimed is:

1. A method for identifying genes responsible for high titer antibody production comprising:
(a) inactivating mismatch repair of said antibody-producing cells, thereby forming hypermutable cells,
(b) screening said hypermutable cells for cells that produce higher titers of antibody as compared to said antibody-producing cells, and
(c) analyzing the genomes of said antibody-producing cells and said hypermutable cells, thereby identifying genes responsible for high titer antibody production.

2. The method of claim 1 wherein said antibody-producing cell produces intact antibodies.

3. The method of claim 1 wherein said antibody-producing cell comprises endogenous immunoglobulin genes.

4. The method of claim 1 wherein said antibody-producing cell comprises exogenous immunoglobulin genes.

5. The method of claim 1 wherein said antibody-producing cell produces derivatives of immunoglobulin genes.

6. The method of claim 1 wherein said step of in activating mismatch repair comprises introducing into said antibody-producing cells a dominant negative allele of a mismatch repair gene.

7. The method of claim 1 wherein said step of in activating mismatch repair comprises knocking out at least one mismatch repair gene of said antibody-producing cells.

8. The method of claim 1 wherein said step of in activating mismatch repair comprises introducing an RNA interference molecule into said antibody-producing cells.

9. The method of claim 1 wherein said step of in activating mismatch repair comprises introducing an antisense molecule against a mismatch repair gene into said antibody-producing cells.

10. The method of claim 6 wherein said allele comprises a truncation mutation.

11. The method of claim 1 wherein the step of screening comprises analyzing a nucleotide sequence of the genome of said cells as compared to the genome of untreated cells.

12. The method of claim 1 wherein the step of screening comprises analyzing mRNA expression levels and structure from said cell as compared to untreated cells.

13. The method of claim 1 wherein the step of testing comprises analyzing protein from the said cell as compared to untreated cells.

14. The method of claim 1 wherein the step of screening comprises analyzing the phenotype of said gene.

15. The method of claim 1 wherein said antibody-producing cell is a mismatch repair defective fertilized egg of a non-human animal.

16. The method of claim 15 further comprising the step of implanting said fertilized egg into a pseudo-pregnant female, whereby said fertilized egg develops into a mature transgenic animal.

17. A homogeneous culture of high titer antibody producing cells produced by a method comprising the steps of:
(a) inactivating mismatch repair of said antibody-producing cells, thereby forming hypermutable cells;
(b) screening said hypermutable cells for cells that produce higher titers of antibody as compared to said antibody-producing cells;
(c) culturing said hypermutable cells producing higher titers of antibody.

18. The culture of high titer antibody producing cells of claim 17 wherein the high titer antibody-producing cell is selected from the group consisting of a bacterial cell, a yeast cell, a plant cell, a mammalian cell, a mouse cell, a rat cell, a rabbit cell, a hamster cell, and a non-human primate cell.

19. A method for producing a high titer antibody producing cell comprising the step of modulating the expression of at least one gene involved in antibody production wherein said genes comprise alpha1-anti-trypsin and endothelial monocyte-activating polypeptide I.

20. The method of claim 19 wherein the cell is a hybridoma.

21. The method of claim 19 where in the cell is an epithelial cell.

22. The method of claim 19 where in the cell is ovarian.

23. The method of claim 19 where in the cell is a kidney cell.

24. The method of claim 19 where in the cell is a myeloid cell.

25. The method of claim 19 where in the cell is a lymphoid cell.

26. The method of claim 19 whereby said step of modulating comprises suppression of the expression of said genes by introducing an antisense oligonucleotide directed against at least one of said endothelial monocyte-activating polypeptide I and alpha-1-anti-trypsin genes.

27. The method of claim 19 whereby said step of modulating comprises suppression of the expression of said genes by introducing an expression vector comprising an antisense transcript directed against at least one of said endothelial monocyte-activating polypeptide I and alpha-1-anti-trypsin genes.

28. The method of claim 19 whereby said step of modulating comprises suppression of the expression of said genes by introducing a knock out targeting vector to disrupt the endogenous function of at least one of said endothelial monocyte-activating polypeptide I and alpha-1-anti-trypsin genes.

29. The method of claim 19 whereby said step of modulating comprises suppression of the expression of said genes by introducing a polynucleotide comprising a ribozyme directed against at least one of said endothelial monocyte-activating polypeptide I and alpha-1-anti-trypsin genes.

30. The method of claim 19 whereby suppression is achieved by introducing intracellular blocking antibodies against the product of said endothelial monocyte-activating polypeptide I and/or the alpha-1-anti-trypsin gene.

31. The method of claim 29 whereby suppression is achieved by incubating cells with neutralizing antibody or derivatives thereof directed against the product of said genes in the growth medium.

32. A method of modulating antibody production of cells comprising contacting said cells with protease inhibitors to decrease antibody production from antibody producer cells.

33. The method of claim 33 where the inhibitor comprises pharmacological amounts of natural protease substrates.

34. The method of claim 33 where said inhibitor is a blocking antibody to natural protease inhibitors.

35. The method of claim 33 where the inhibitor is a blocking antibody to alpha-1-anti-trypsin.

36. A method for selecting cells for high titer antibody production whereby growth medium of cells is analyzed for alpha-1-antitrypsin, where low levels are associated with high antibody titers.

37. The method of claim 36 wherein alpha-1-antitrypsin RNA, wherein low levels of RNA is associated with high antibody titers.

38. The method of claim 36 wherein alpha-1-antitrypsin protein, wherein low levels of RNA is associated with high antibody titers.

39. A method for selecting for cells for high titer antibody production whereby growth medium of cells is analyzed for endothelial monocyte-activating polypeptide I, where low levels are associated with high antibody titers.

40. The method of claim 39 wherein endothelial monocyte-activating polypeptide I RNA, wherein low levels of RNA is associated with high antibody titers.

41. The method of claim 39 wherein endothelial monocyte-activating polypeptide I protein, wherein low levels of RNA is associated with high antibody titers.

42. A method for suppressing antibody production associated with hyperimmunoglobulin disease production comprising contacting said cells with at least one compound that increases endothelial monocyte-activating polypeptide I expression.

43. A method for suppressing antibody production associated with hyperimmunoglobulin disease production comprising contacting said cells with at least one compound that increases alpha-1-antitrypsin expression.

44. A method for enhancing antibody production associated with hypoimmunoglobulin disease production comprising contacting said cells with at least one compound that suppresses alpha-1-anti-trypsin expression activity.

45. The method of claim 44 wherein said compound decreases the activity of alpha-1-antitrypsin protein in said cells.

46. The method of claim 44 wherein said compound decreases the level of alpha-1-antitrypsin in said cells.

47. A method for enhancing antibody production associated with hypoimmunoglobulin disease production comprising contacting said cells with at least one compound that suppresses monocyte-activating polypeptide I expression activity.

48. The method of claim 47 wherein said compound decreases the activity of monocyte-activating polypeptide I protein in said cells.

49. The method of claim 47 wherein said compound decreases the level of monocyte-activating polypeptide I in said cells.

50. A host cell for the expression of antibody molecules or fragments thereof comprising a defect in the monocyte-activating polypeptide I gene such that expression of monocyte-activating polypeptide I is inhibited.

51. The host cell of claim 50 wherein said defect comprises a deletion of the monocyte-activating polypeptide I.

52. The host cell of claim 50 wherein said defect is a frameshift mutation in the monocyte-activating polypeptide I gene.

53. The host cell of claim 50 wherein said host cell comprises an expression vector comprising an antisense transcript of the monocyte-activating polypeptide I gene whereby expression of said antisense transcript suppresses the expression of the monocyte-activating polypeptide I gene.

54. The host cell of claim 50 wherein said host cell comprises a ribozyme that disrupts expression of the monocyte-activating polypeptide I gene.

55. The host cell of claim 50 wherein said host cell comprises an intracellular neutralizing antibody against the monocyte-activating polypeptide I protein whereby said antibody suppresses the activity of monocyte-activating polypeptide I.

56. A host cell for the expression of antibody molecules or fragments thereof comprising a defect in the alpha-1-antitrypsin gene such that expression of alpha-1-antitrypsin is inhibited.

57. The host cell of claim 56 wherein said defect comprises a deletion of the alpha-1-antitrypsin.

58. The host cell of claim 56 wherein said defect is a frameshift mutation in the alpha-1-antitrypsin gene.

59. The host cell of claim 56 wherein said host cell comprises an expression vector comprising an antisense transcript of the alpha-1-antitrypsin gene whereby expression of said antisense transcript suppresses the expression of the alpha-1-antitrypsin gene.

60. The host cell of claim 56 wherein said host cell comprises a ribozyme that disrupts expression of the alpha-1-antitrypsin gene.

61. The host cell of claim 56 wherein said host cell comprises an intracellular neutralizing antibody against the alpha-1-antitrypsin protein whereby said antibody suppresses the activity of alpha-1-antitrypsin.

62. The host cell of claim 61 further comprising an expression vector comprising a polynucleotide sequence encoding at least a portion of an antibody molecule.

63. The host cell of claim 61 wherein said polynucleotide encodes at least an immunoglobulin light chain or fragment thereof.

64. The host cell of claim 61 wherein said polynucleotide encodes at least an immunoglobulin heavy chain or fragment thereof.

65. The method of claim 1 further comprising the step of restabilizing the genome of selected high titer antibody-producing cells.

66. A culture of stable, high titer antibody-producing cells made by the method of claim 65.
